SEQUENCE LISTING (71 sequences)

SEQ ID NO: 1 - RNA, 81 nt, Artificial Sequence
  Wild-type pre-mRNA sequence of human COL7A1 exon 105
ggauccccaggaaaggauggagugccugguauccgaggagaaaaaggagauguuggcuucauggguccccggggccucaag

SEQ ID NO: 2 - RNA, 96 nt, Artificial Sequence
  Human COL7A1 pre-mRNA fragment including wild-type exon 105
ucucccugcuagggauccccaggaaaggauggagugccugguauccgaggagaaaaaggagauguuggcuucauggguccccggggccucaaggua

SEQ ID NO: 3 - RNA, 20 nt, Artificial Sequence
  AON UMCG-AON1
gauaccaggcacuccauccu

SEQ ID NO: 4 - RNA, 20 nt, Artificial Sequence
  AON UMCG-AON2
caugaagccaacaucuccuu

SEQ ID NO: 5 - RNA, 20 nt, Artificial Sequence
  AON 1
acaucuccuuuuucuccucg

SEQ ID NO: 6 - RNA, 20 nt, Artificial Sequence
  AON 1b
uccuuuuucuccucggauac

SEQ ID NO: 7 - RNA, 20 nt, Artificial Sequence
  AON 2
ggcacuccauccuuuccugg

SEQ ID NO: 8 - RNA, 20 nt, Artificial Sequence
  AON 2b
aggcacuccauccuuuccug

SEQ ID NO: 9 - RNA, 20 nt, Artificial Sequence
  AON 3
ccaugaagccaacaucuccu

SEQ ID NO: 10 - RNA, 18 nt, Artificial Sequence
  AON 4
ggaucccuagcagggaga

SEQ ID NO: 11 - RNA, 20 nt, Artificial Sequence
  AON 5
uucuuuccuaccuugaggcc

SEQ ID NO: 12 - RNA, 20 nt, Artificial Sequence
  AON 6
cuuuuucuccucggauacca

SEQ ID NO: 13 - RNA, 24 nt, Artificial Sequence
  AON 7
cuccucggauaccaggcacuccau

SEQ ID NO: 14 - RNA, 24 nt, Artificial Sequence
  AON 8
ccgggacccaugaagccaacaucu

SEQ ID NO: 15 - RNA, 20 nt, Artificial Sequence
  AON 9
ccgggacccaugaagccaac

SEQ ID NO: 16 - RNA, 20 nt, Artificial Sequence
  AON 10/35
gacccaugaagccaacaucu

SEQ ID NO: 17 - RNA, 20 nt, Artificial Sequence
  AON 11
gggacccaugaagccaacau

SEQ ID NO: 18 - RNA, 24 nt, Artificial Sequence
  AON 12
cggggacccaugaagccaacaucu

SEQ ID NO: 19 - RNA, 24 nt, Artificial Sequence
  AON 13
cccgggacccaugaagccaacauc

SEQ ID NO: 20 - RNA, 23 nt, Artificial Sequence
  AON 14
ccgggacccaugaagccaacauc

SEQ ID NO: 21 - RNA, 20 nt, Artificial Sequence
  AON 15
cggggacccaugaagccaac

SEQ ID NO: 22 - RNA, 20 nt, Artificial Sequence
  AON 16
ccggggacccaugaagccaa

SEQ ID NO: 23 - RNA, 22 nt, Artificial Sequence
  AON 17
ccgggacccaugaagccaacau

SEQ ID NO: 24 - RNA, 24 nt, Artificial Sequence
  AON 18
gacccaugaagccaacaucuccuu

SEQ ID NO: 25 - RNA, 24 nt, Artificial Sequence
  AON 19
ggccccgggacccaugaagccaac

SEQ ID NO: 26 - RNA, 24 nt, Artificial Sequence
  AON 20
gggacccaugaagccaacaucucc

SEQ ID NO: 27 - RNA, 21 nt, Artificial Sequence
  AON 21
ccaugaagccaacaucuccuu

SEQ ID NO: 28 - RNA, 21 nt, Artificial Sequence
  AON 22
cccaugaagccaacaucuccu

SEQ ID NO: 29 - RNA, 21 nt, Artificial Sequence
  AON 23
acccaugaagccaacaucucc

SEQ ID NO: 30 - RNA, 21 nt, Artificial Sequence
  AON 24
gacccaugaagccaacaucuc

SEQ ID NO: 31 - RNA, 21 nt, Artificial Sequence
  AON 25
ggacccaugaagccaacaucu

SEQ ID NO: 32 - RNA, 21 nt, Artificial Sequence
  AON 26
gggacccaugaagccaacauc

SEQ ID NO: 33 - RNA, 21 nt, Artificial Sequence
  AON 27
cgggacccaugaagccaacau

SEQ ID NO: 34 - RNA, 21 nt, Artificial Sequence
  AON 28
ccgggacccaugaagccaaca

SEQ ID NO: 35 - RNA, 22 nt, Artificial Sequence
  AON 29
gacccaugaagccaacaucucc

SEQ ID NO: 36 - RNA, 22 nt, Artificial Sequence
  AON 30
ggacccaugaagccaacaucuc

SEQ ID NO: 37 - RNA, 22 nt, Artificial Sequence
  AON 31
gggacccaugaagccaacaucu

SEQ ID NO: 38 - RNA, 23 nt, Artificial Sequence
  AON 32
ggacccaugaagccaacaucucc

SEQ ID NO: 39 - RNA, 23 nt, Artificial Sequence
  AON 33
gggacccaugaagccaacaucuc

SEQ ID NO: 40 - RNA, 19 nt, Artificial Sequence
  AON 34
cauccuuuccuggggaucc

SEQ ID NO: 41 - RNA, 20 nt, Artificial Sequence
  AON 36
ggacccaugaagccaacauc

SEQ ID NO: 42 - RNA, 19 nt, Artificial Sequence
  AON 37
ccaugaagccaacaucucc

SEQ ID NO: 43 - RNA, 20 nt, Artificial Sequence
  AON 38
cccaugaagccaacaucucc

SEQ ID NO: 44 - RNA, 21 nt, Artificial Sequence
  AON 39
cuccucggauaccaggcacuc

SEQ ID NO: 45 - RNA, 18 nt, Artificial Sequence
  AON 40
ccaugaagccaacaucuc

SEQ ID NO: 46 - RNA, 20 nt, Artificial Sequence
  AON 41
guuucuuuccuaccuugagg

SEQ ID NO: 47 - RNA, 18 nt, Artificial Sequence
  AON 42
ggacccaugaagccaaca

SEQ ID NO: 48 - RNA, 18 nt, Artificial Sequence
  AON 43
caugaagccaacaucucc

SEQ ID NO: 49 - RNA, 20 nt, Artificial Sequence
  AON 44
uucuccucggauaccaggca

SEQ ID NO: 50 - RNA, 20 nt, Artificial Sequence
  AON 45
ucuccucggauaccaggcac

SEQ ID NO: 51 - RNA, 20 nt, Artificial Sequence
  AON 46
ccucggauaccaggcacucc

SEQ ID NO: 52 - RNA, 20 nt, Artificial Sequence
  AON 47
ccaggcacuccauccuuucc

SEQ ID NO: 53 - RNA, 20 nt, Artificial Sequence
  AON 48
ggauaccaggcacuccaucc

SEQ ID NO: 54 - RNA, 20 nt, Artificial Sequence
  AON 49
cuuguuucuuuccuaccuug

SEQ ID NO: 55 - RNA, 20 nt, Artificial Sequence
  AON 50
uguuucuuuccuaccuugag

SEQ ID NO: 56 - RNA, 19 nt, Artificial Sequence
  AON 51
caugaagccaacaucuccu

SEQ ID NO: 57 - RNA, 19 nt, Artificial Sequence
  AON 52
augaagccaacaucuccuu

SEQ ID NO: 58 - RNA, 19 nt, Artificial Sequence
  AON 53
ccucggauaccaggcacuc

SEQ ID NO: 59 - RNA, 20 nt, Artificial Sequence
  AON 54
cggauaccaggcacuccauc

SEQ ID NO: 60 - RNA, 18 nt, Artificial Sequence
  AON 55
cucggauaccaggcacuc

SEQ ID NO: 61 - DNA, 13 bp, Artificial Sequence
  COL7A1 pre-mRNA exon 105 region 1 (E105-R1)
ttggcttcatggg

SEQ ID NO: 62 - RNA, 13 nt, Artificial Sequence
  AON sequence targeting E105-R1
cccaugaagccaa

SEQ ID NO: 63 - DNA, 19 bp, Artificial Sequence
  cDNA Forward primer; C7-del105 Forward primer
gtgacaaaggacctcgggg

SEQ ID NO: 64 - DNA, 19 bp, Artificial Sequence
  cDNA Reverse primer; C7-del105 Reverse primer
ctccatcaaggccacaggc

SEQ ID NO: 65 - DNA, 22 bp, Artificial Sequence
  GusB Forward primer
gtttttgatccagacccagatg

SEQ ID NO: 66 - DNA, 21 bp, Artificial Sequence
  GusB Reverse primer
gcccattattcagagcgagta

SEQ ID NO: 67 - DNA, 23 bp, Artificial Sequence
  GusB Probe (HEX)
tgcagggtttcaccaggatccac

SEQ ID NO: 68 - DNA, 18 bp, Artificial Sequence
  C7 Forward primer
tcggttgctggaaactgc

SEQ ID NO: 69 - DNA, 18 bp, Artificial Sequence
  C7 Reverse primer
cacaggcaggaagctacc

SEQ ID NO: 70 - DNA, 24 bp, Artificial Sequence
  C7 Probe (FAM)
atcaaggcatctgccctgcgggag

SEQ ID NO: 71 - DNA, 22 bp, Artificial Sequence
  C7-del105 Probe (FAM)
actccccgttcacccgggtcac

(19) United States
(12) Patent Application Publication
(10) Pub. No.: US 2022/0127610 A1
(43) Pub. Date: Apr. 28, 2022

(54) OLIGONUCLEOTIDES FOR USE IN THE TREATMENT OF DYSTROPHIC EPIDERMOLYSIS BULLOSA

(71) Applicant: Phoenicis Therapeutics, Inc., Hingham, MA (US)

(72) Inventors: Jim Swildens, Hingham, MA (US); Elisabeth Marlene Haisma, Hingham, MA (US)

(73) Assignee: Phoenicis Therapeutics, Inc., Hingham, MA (US)

(21) Appl. No.: 17/434,727

(22) PCT Filed: Feb. 28, 2020
(86) PCT No.: PCT/US2020/020541

§ 371 Date: Aug. 27, 2021

(30) Foreign Application Priority Data
Feb. 28, 2019 (GB) 1902735.8

(51) Int. Cl. C12N 15/113

(52) U.S. Cl. CPC C12N 15/113; C12N 2310/11; C12N 2310/315; C12N 2310/321; C12N 2320/33; C12N 2310/344; C12N 2310/346; C12N 2310/3521; C12N 2310/3525; C12N 2310/3231

(57) ABSTRACT

The invention relates to antisense oligonucleotides (AONs) and the use thereof in the treatment of human disease. In particular the present invention is concerned with AONs suitable for the treatment of Dystrophic Epidermolysis Bullosa (DEB). More specifically, the invention relates to AONs that are capable of inducing exon 105 skipping from human COL7A1 pre-mRNA and that outperform—in exon 105 skipping efficiency—the AONs known from the prior art. The invention further relates to the use of these new AONs in the treatment of DEB in a patient carrying a mutation in exon 105 of the COL7A1 gene.

### CROSS-REFERENCE TO RELATED APPLICATIONS

This application claims the benefit of priority to United Kingdom Patent Application No. 1902735.8, filed Feb. 28, 2019, the disclosure of which is incorporated herein by reference in its entirety.

### INCORPORATION OF THE SEQUENCE LISTING

The material in the accompanying Sequence Listing is hereby incorporated by reference into this application. The accompanying Sequence Listing text file, named 056520-503001WO_Sequence_Listing_ST25.TXT, was created on Feb. 28, 2020, and is 14 KB in size.

### FIELD OF THE INVENTION

The present invention relates to antisense oligonucleotides (AONs) and the use thereof in the treatment of human disease. In particular the present invention is concerned with AONs suitable for the treatment of Dystrophic Epidermolysis Bullosa (DEB). More specifically, the invention relates to AONs that are capable of inducing exon 105 skipping from human COL7A1 pre-mRNA, and their use in the treatment of DEB in a patient carrying a mutation in exon 105 of the COL7A1 gene.

### BACKGROUND OF THE INVENTION

Epidermolysis Bullosa (EB) is a group of heritable skin diseases, which are characterized by chronic fragility and blistering of the skin and mucous membranes. Depending on the subtype, the spectrum of symptoms of the EB is very broad, ranging from minimal skin fragility to very severe symptoms with general complications. Worldwide about 350,000 patients are affected. In some forms of EB, also nails, hair and teeth may be involved. The main types of EB include EB Simplex (EBS), Junctional EB (JEB), Dystrophic EB (DEB) and Kindler syndrome (KS).

DEB affects approximately 44,000 patients worldwide. Blistering and skin erosions occur upon the slightest touch or even occur spontaneously. Symptoms include open wounds, skin infections and fusion of fingers and toes (pseudo syndactyly). Patients with Recessive DEB (RDEB; approximately 50% of DEB patients) suffer from lifelong generalized blistering, chronic ulcerations and scarring sequelae, leading to multi organ involvement, major morbidity, life threating complications and squamous cell carcinoma (SCC). The occurrence of SCC reduces life expectancy in patients with RDEB; they are unlikely to survive beyond the 3rd or 4th decade of their lives. Characteristics of Dominant DEB (DDEB) include blistering that may be localized to the hands, feet, elbows and knees or generalized. Common findings include scarring, milia, mucous membrane involvement, and abnormal or absent nails. RDEB is typically more generalized and severe than DDEB. In addition to the symptoms of DDEB, common manifestations of RDEB include malnutrition, anemia, osteoporosis, esophageal strictures, growth retardation, webbing, or fusion of the fingers and toes causing mitten deformity, development of muscle contractures, malformation of teeth, microstomia and scarring of the eye.

Despite the high unmet medical need there is currently no disease modifying treatment available for DEB patients; only palliative care is performed. Severe forms of RDEB impose a high cost on society's healthcare budget: the average costs of dressings and medication is about €200,000 per patient per year.

DEB is caused by one or more mutations in the COL7A1 gene that codes for Type VII collagen alpha 1 protein (C7). C7 is the main component of anchoring fibrils (AFs) that link the dermis to the epidermis. AFs form by the trimerization of C7-alpha chains. These trimers subsequently assemble into antiparallel-filaments and these in turn interact at the N-terminal domain with laminin-332 and Type IV collagen within the lamina densa zone of the basement membrane. Decreased levels of functional C7 therefore lead to absent or malfunctioning anchoring fibrils which then leads to skin fragility. DEB disease severity roughly correlates with the amount of Type VII collagen expression at the basement membrane zone.

Within the COL7A1 gene more than 400 different mutations are known that include missense mutations and mutations leading to ‘premature termination codons’ (PTCs). The human COL7A1 gene contains 118 exons. The majority of these are in-frame, which means that if that particular exon would not be present the neighboring exons (when linked together) would still be translated in-frame. Exons 1, 2, 3, 4, 6, 7, 24, 25, 27, 113 and 118 are not in-frame. Because the in-frame exons harbor many mutations causing DEB, exon skipping of such in-frame exons was identified as a potentially viable method to get rid of the mutations, while retaining protein function (Goto et al. 2006. Targeted Skipping of a Single Exon Harboring a Premature Termination Codon Mutation: Implications and Potential for Gene Correction Therapy for Selective Dystrophic Epidermolysis Bullosa Patients. J Invest Dermatol 126(12):2614-2620), and this strategy has been further explored by several research groups. In fact, exon skipping in COL7A1 pre-mRNA by applying AONs has been suggested and/or shown for exon 13, 73, 74, 80 and 105 (Bornert et al. 2016. Analysis of the functional consequences of targeted exon deletion in COL7A1 reveals prospects for dystrophic epidermolysis bullosa therapy. Mol Ther 24(7):1302-1311; Bremer et al. 2016 Antisense Oligonucleotide-mediated Exon Skipping as a Systemic Therapeutic Approach for Recessive Dystrophic Epidermolysis Bullosa. Mol Ther Nucleic Acids 5(10):e379; Goto et al. 2006; Turczynski et al. 2016. Targeted Exon Skipping Restores Type VII Collagen Expression and Anchoring Fibril Formation in an In Vivo RDEB Model. J Invest Dermatol 136(12):2387-2395; WO2013/053819; WO2016/142538; WO2016/185041; WO2017/078526). For C7 lacking exon 13 or 105 it was demonstrated that the protein folding was not affected and that cell adhesion and migration processes (in which C7 plays a role) were normal (Bornert et al. 2016). Moreover, for exon 73 and 80 it was shown that the slightly shorter C7Δ73 and C7Δ80 proteins produced by patient cells after skipping of these exons, respectively, could be incorporated at the dermal-epidermal junction and form anchoring fibrils (Turczynski et al. 2016). The intended route of AON administration is through topical application using a hydrogel vehicle.

Although the exon skipping oligonucleotides of the prior art provide a promising step in tackling this terrible disease, there is still a need for further alternative oligonucleotides that improve the efficiency of exon skipping. Notably, the skipping of exon 105 was only found to be effective when two AONs were used in combination (Bornert et al. 2016; Bremer et al. 2016; WO2017/078526). The present invention aims to identify and use improved AONs for exon 105 skipping from COL7A1 pre-mRNA, that outperform the AONs of the prior art, and that can preferably be used as a single active ingredient.

### SUMMARY OF THE INVENTION

The invention provides various antisense oligonucleotides (AONs) that are capable of preventing or reducing exon 105 inclusion into a human collagen type VII alpha 1 chain (COL7A1) mRNA, when the mRNA is produced by splicing from a pre-mRNA in a mammalian cell (such as in a human cell in vivo). In one aspect, the invention relates to an AON capable of preventing or reducing exon 105 inclusion into a human COL7A1 mRNA when the mRNA is produced by splicing from a pre-mRNA in a cell, wherein the AON comprises or consists of a nucleotide sequence that is selected from the group consisting of SEQ ID NOs: 48, 42, 60, 44, 46, 51, 52, 58, 59, 5-41, 43, 45, 47, 49, 50, and 53-57. In some embodiments, the AON comprises or consists of a nucleotide sequence that is selected from the group consisting of SEQ ID NOs: 48, 42, 60, 44, 46, 51, 52, 58, and 59. In some embodiments, the AON comprises or consists of a nucleotide sequence that is selected from the group consisting of SEQ ID NOs: 48, 42, and 60. In some embodiments, the AON comprises or consists of a nucleotide sequence that is selected from the group consisting of SEQ ID NOs: 24, 26, 14, 15, 16, 17, 18, 19, 20, 21, 22, 23, 25, 28, 29, 30, 31, 32, 33, 34, 35, 36, 37, 38, 39, 5, 6, 8, 9, 10, 11, 12, and 13. Preferred AONs of the present invention consist of a nucleotide sequence according to SEQ ID NO: 24, SEQ ID NO: 26 or SEQ ID NO: 14. In another aspect, the invention relates to an AON capable of preventing or reducing exon 105 inclusion into a human COL7A1 mRNA when the mRNA is produced by splicing from a pre-mRNA in a cell, wherein the AON comprises or consists of a nucleotide sequence that is complementary to a target nucleotide sequence in a COL7A1 pre-mRNA corresponding to any one of SEQ ID NOs: 48, 42, 60, 44, 46, 51, 52, 58, 59, 5-41, 43, 45, 47, 49, 50, and 53-57. In some embodiments, the AON comprises or consists of a nucleotide sequence that is complementary to a target nucleotide sequence in a COL7A1 pre-mRNA corresponding to any one of SEQ ID NOs: 48, 42, 60, 44, 46, 51, 52, 58, and 59. In some embodiments, the AON comprises or consists of a nucleotide sequence that is complementary to a target nucleotide sequence in a COL7A1 pre-mRNA corresponding to SEQ ID NO: 48, 42, or 60. In some embodiments, the AON comprises or consists of a nucleotide sequence that is complementary to a target nucleotide sequence in a COL7A1 pre-mRNA corresponding to any one of SEQ ID NOs: 24, 26, 14, 15, 16, 17, 18, 19, 20, 21, 22, 23, 25, 28, 29, 30, 31, 32, 33, 34, 35, 36, 37, 38, 39, 5, 6, 8, 9, 10, 11, 12, and 13. In a preferred embodiment, the AON of the present invention is 18, 19, 20, 21, 22, 23, 24, 25, 26, 27, or 28 nucleotides in length. More preferably, the AON according to the present invention is 24 nucleotides in length. In yet another preferred aspect, the AON according to the invention comprises at least one non-natural linkage modification, preferably a phosphorothioate linkage. In one particularly preferred aspect, all sugar moieties of the AON according to the present invention are modified with a 2′-O-methyl (2′-OMe) substitution, or with a 2′-methoxyethoxy (2′-MOE) substitution. In another aspect, the 5′- and 3′-terminal nucleotides are LNA-modified, and all other sugar moieties are modified with a 2′-OMe substitution or a 2′-MOE substitution. In yet another aspect, the two 5′- and two 3′-terminal nucleotides are LNA-modified, and all other sugar moieties are modified with a 2′-OMe substitution or a 2′-MOE substitution.

In another embodiment, the invention relates to a viral vector comprising a nucleotide sequence encoding an AON according to the invention. The invention also relates to a pharmaceutical composition comprising an AON, or a viral vector according to the invention, wherein the composition further comprises one or more of a carrier, excipient, stabilizer, transfection agent, diluent, gelling agent or buffer.

In another aspect, the invention relates to a method of preventing or reducing exon 105 inclusion into a human COL7A1 mRNA when the mRNA is produced by splicing from a pre-mRNA in a human cell, the method comprising providing the cell in an in vitro, in vivo or ex vivo setting, and administering to the cell an AON according to the invention, a viral vector according to the invention, or a pharmaceutical composition according to the invention.

In yet another aspect, the invention relates to an AON according to the invention for use in the treatment of a human subject suffering from dystrophic epidermolysis bullosa (DEB), preferably wherein the DEB is caused by a mutation in exon 105 of the COL7A1 gene in the subject, more preferably wherein the mutation is a c.7864delC mutation. The treatment preferably comprises topical administration of the AON, e.g., in a hydrogel formulation, such as a carbomer hydrogel formulation. In another aspect, the invention relates to use of an AON according to the invention or a viral vector according to the invention in the manufacture of a medicament for the treatment, prevention, amelioration or delay of DEB in a human subject. In another aspect, the invention relates to a method for the treatment of DEB in a human subject, comprising the step of administering to the subject an AON according to the invention, a viral vector according to the invention, or a pharmaceutical composition according to the invention. In some embodiments, the DEB is caused by a mutation in exon 105 of a COL7A1 gene in the human subject. In some embodiments, the mutation is a c.7864delC mutation.

### BRIEF DESCRIPTION OF THE DRAWINGS

FIG. 1 shows—on top—a wild type (here RNA) sequence of exon 105 (5′ to 3; upper case, bold; SEQ ID NO: 1) of the human COL7A1 gene with part of the upstream and downstream intron sequences (lower case). The position of the c.7864delC mutation is underlined. Below this intron-exon-intron sequence (SEQ ID NO: 2) the respective sequences of the antisense oligonucleotides (AONs) with their corresponding identifiers and SEQ ID NOs are given from 3′ to 5′ (left to right). The overlap with the wild type sequence, in respect of the position of the c.7864delC mutation, is given with an underlined guanosine in AONs 12, 15 and 16. Where the AON is overlapping that particular position, but lacks a guanosine, it is indicated by “-”, as in AONs 8, 9, 13, 14, 17, 19, 27 and 28.

FIG. 2 shows the PCR results on cDNA from mRNA obtained from wild type fibroblasts transfected with two different AONs known from the art (herein referred to as “UMCG-AON1” or “UMCG-1” and “UMCG-AON2” or “UMCG-2”) in two modified versions, one in which the AON is fully modified with 2′-OMe substitutions in the sugar moiety (here depicted as “2 o-ME”) and one in which the AON is fully modified with 2′-MOE modifications in the sugar moiety (here depicted as “MOE”). These AONs were compared with one new AON described in detail herein (AON8), which was fully 2′-MOE-modified. The arrows on the right indicate the size of the PCR product with the used primers, indicating the position of the wt product (upper arrow) and the product in which exon 105 has been skipped and is not present in the generated cDNA (lower arrow). NT is a Not-Transfected negative control and PEI is a negative control with transfection reagent only.

FIG. 3 shows the PCR results on cDNA from mRNA obtained from wild type fibroblasts transfected with five different AONs (AON7, -8, -9, -10, and -11), each in a 2′-OMe (here “OMe”) and a 2′-MOE (here “MOE”) version. The arrows indicate the size of the PCR product with the used primers, indicating the position of the wt product (above) and the product in which exon 105 has been skipped and is not present in the generated cDNA (below). M is the marker, NT is a Not-Transfected negative control and PEI is a negative control with transfection reagent only.

FIG. 4 shows the PCR results from mRNA and cDNA obtained from wild type fibroblasts transfected with AON12, -13, -14, -15, -16, -17, -18, -19, and -20, all fully modified with 2′-MOE in the sugar moiety. AON8 in a 2′-MOE (here “MOE”) and 2′-OMe version (here “20-Me”; carried out in duplicate) were included. The arrows on the right indicate the presence of exon 105 (upper arrow) or absence of exon 105 (lower arrow) in the obtained mRNA.

FIG. 5 shows the percentage of exon 105 skipping using a ddPCR assay, after transfection of AON8, -12, -15, -16, -18, and -20 all in a 2′-OMe version (here “2oMe”) and in a 2′-MOE version (here “MOE”) in wild type human fibroblasts. Four negative controls were included: NaCl only, transfection reagent only (MaxPEI), water only, and transfection with a non-related 2′-MOE-modified scrambled oligonucleotide (MOE Scr).

FIG. 6 shows the percentage of COL7A1 (C7) exon 105 skipping using a ddPCR assay, after transfection of AON8, AON18 and AON20, all three in a 2′-OMe version (here “2OMe”) and in a 2′-MOE version (here “MOE”) in wild type human fibroblasts (FD030), fibroblasts obtained from a DEB patient suffering from the c.7864delC mutation (PLU002A), and fibroblasts obtained from a human subject carrying the c.7864delC deletion in one allele but not suffering from DEB (PLU003A). Four negative controls were included: NaCl only, transfection reagent only (MaxPEI), a reverse transcription control (RT-CTRL) and transfection with a non-related 2′-MOE-modified scrambled oligonucleotide (MOE Scr).

FIG. 7 shows the percentage of COL7A1 (C7) exon 105 skipping using a ddPCR assay, after transfection of AON21, -22, -23, -24, -25, -26, -27, -28, -29, -30, -31, -32, and -33, all fully modified with 2′-OMe (here “2OMe”) in PLU002A fibroblasts and in PLU003A fibroblasts in duplicate (P6 and P7). Positive controls were: AON8 that was fully modified with 2′-MOE; and AON18 in two versions: 2′-MOE and 2′-OMe, in two concentrations (100 and 250 nM). Five negative controls were included: NaCl only, transfection reagent only (MaxPEI), a reverse transcription control (RT-CTRL), a transfection with a non-related 2′-MOE-modified scrambled oligonucleotide (MOE Scr), and a water only control.

FIG. 8 shows the percentage of exon 105 skipping using a ddPCR assay, after transfection of 2′-OMe and 2′-MOE versions of AON8, AON18, AON20, AON23, AON29 and AON32 in PLU002A and in PLU003A fibroblasts. The three negative controls were included: NaCl only, MaxPEI, and a scrambled 2′-MOE-modified oligonucleotide.

FIG. 9 shows the percentage of exon 105 skipping using a ddPCR assay, after transfection of the 2′-OMe (here “2OMe”) and 2′-MOE (here “MOE”) versions of AON8, AON18 and AON20 in PLU002A fibroblasts. The skipping efficiency of these three best performing AONs was compared to the skipping efficiency of the AONs from the prior art: UMCG-AON1 and UMCG-AON2 (see Table 1), that were transfected in PLU002A cells either alone or in combination. Both UMCG AONs were tested in the full 2′-OMe and in the full 2′-MOE versions, as depicted. Negative controls were NaCl and transfection reagent only (MaxPEI).

FIG. 10 shows the frequency of exon 105 skipping in human COL7A1 pre-mRNA from human fibroblasts transfected with T-OMe variants of the indicated AONs, as assayed by ddPCR. 1×LNA: 5′- and 3′-terminal nucleotides LNA-modified, all other nucleotides 2′-OMe-modified. 2×LNA: two 5′- and two 3′-terminal nucleotides LNA-modified, all other nucleotides T-OMe-modified. Negative controls: NaCl and transfection reagent only (MaxPEI).

FIG. 11 shows the frequency of exon 105 skipping in human COL7A1 pre-mRNA from human fibroblasts transfected with T-MOE variants of the indicated AONs, as assayed by ddPCR. 1×LNA: 5′- and 3′-terminal nucleotides LNA-modified, all other nucleotides 2′-MOE-modified. 2×LNA: two 5′- and two 3′-terminal nucleotides LNA-modified, all other nucleotides 2′-MOE-modified. Negative controls: NaCl and transfection reagent only (MaxPEI).

FIG. 12 shows the frequency of exon 105 skipping in human COL7A1 pre-mRNA from HeLa cells following gymnotic uptake of 2′-OMe variants of the indicated AONs, as assayed by ddPCR. 1×LNA: 5′- and 3′-terminal nucleotides LNA-modified, all other nucleotides 2′-OMe-modified. 2×LNA: two 5′- and two 3′-terminal nucleotides LNA-modified, all other nucleotides 2′-OMe-modified. Negative control: NaCl.

FIG. 13 shows the frequency of exon 105 skipping in human COL7A1 pre-mRNA from HeLa cells following gymnotic uptake of 2′-MOE variants of the indicated AONs, as assayed by ddPCR. 1×LNA: 5′- and 3′-terminal nucleotides LNA-modified, all other nucleotides T-MOE-modified. 2×LNA: two 5′- and two 3′-terminal nucleotides LNA-modified, all other nucleotides T-MOE-modified. Negative control: NaCl.

FIG. 14 shows dose-response for the indicated AONs at various concentrations (3 μM, 10 μM, 30 μM, and 50 μM) on the frequency of exon 105 skipping in human COL7A1 pre-mRNA from HeLa cells following gymnotic uptake of the AONs, as assayed by ddPCR. OMe: all nucleotides 2′-OMe-modified. OMe/1×LNA: 5′- and 3′-terminal nucleotides LNA-modified, all other nucleotides 2′-OMe-modified. MOE/1×LNA: 5′- and 3′-terminal nucleotides LNA-modified, all other nucleotides T-MOE-modified. Negative control: non-treated.

FIG. 15 shows the frequency of exon 105 skipping in human COL7A1 pre-mRNA from dermis, epidermis, or both dermis and epidermis of human skin equivalent (HSE) models of wounding following treatment for three weeks with the indicated AONs in a carbomer hydrogel formulation, as assayed by ddPCR. OMe: all nucleotides 2″-OMe-modified. OMe/1×LNA: 5′- and 3′-terminal nucleotides LNA-modified, all other nucleotides 2′-OMe-modified. MOE/1×LNA: 5′- and 3′-terminal nucleotides LNA-modified, all other nucleotides 2′-MOE-modified. Negative control: non-treated.

### DETAILED DESCRIPTION OF THE INVENTION

The present invention discloses antisense oligonucleotides (AONs) that appear to have similar or better exon 105 skipping characteristics when compared to those disclosed in the prior art. The AONs of the present invention can be used as active drug substances in therapies to treat human disease, more in particular Epidermolysis Bullosa (EB), even more in particular EB associated with mutations in exon 105 of the human COL7A1 gene. The AONs of the present invention are preferably used as sole active drug substance, but may also be used in combination with other AONs targeting COL7A1 exon 105 (including the ones from the prior art and/or those disclosed herein), in combination with AONs targeting other exons (in the case of double mutants, either present on the same allele or on different alleles, including mutations in exons 13, 73, 74 or 80) and/or in combination with other active drug substances for treating EB disease. Combination therapy may be in the form of a single composition or multiple compositions, administered simultaneously or consecutively.

Several exon 105 mutations have been identified in the art, which in principle can all be removed by exon 105 skipping using an AON disclosed for the first time herein. Examples of mutations that were previously identified to be present in exon 105 of human COL7A1 are c.7795G>T, c.7804G>A, c.7805G>A, c.7828C>T, c.7856del1, c.7861_7865del5, c.7864C>T, c.7864delC, c.7865G>A, c.7868G>A, c.7868G>T, and c.7875+1G>C (Escamez et al. 2010. The first COL7A1 mutation survey in a large Spanish dystrophic epidermolysis bullosa cohort: c.6527insC disclosed as an unusually recurrent mutation. Br J Dermatol 163 (1): 155-161; Varki et al. 2007. Epidermolysis bullosa. II. Type VII collagen mutations and phenotype-genotype correlations in the dystrophic subtypes. J Med Genet 44:181-192; col7a1-database.info). The c.7864delC mutation (protein: p.Arg2622GlyfsX9) is further used herein as a non-limiting example.

As disclosed herein, AONs have been identified that are capable of preventing or reducing exon 105 inclusion into a human COL7A1 mRNA, when the mRNA is produced by splicing from a pre-mRNA in a mammalian cell, characterized in that the oligonucleotide's sequence is, preferably 100%, complementary to an internal part of exon 105. AONs are described that are capable of preventing or reducing exon 105 inclusion into a human COL7A1 mRNA when the mRNA is produced by splicing from a pre-mRNA in a mammalian cell. These AONs are considered good candidates to be used in preventing or reducing exon 105 inclusion into a human COL7A1 mRNA. The AONs of the present invention may be used in combination with each other or other AONs useful for skipping exon 105 (or other in-frame exons in the human COL7A1 mRNA), but preferably the AONs of the present invention are used a sole active compound in a medicament for the treatment of DEB caused by a mutation in exon 105.

In one aspect, the invention relates to an AON capable of preventing or reducing exon 105 inclusion into a human COL7A1 mRNA when the mRNA is produced by splicing from a pre-mRNA in a cell, wherein the AON comprises or consists of a nucleotide sequence that is selected from the group consisting of SEQ ID NOs: 5-60 and variants thereof that confer at least some (such as all or substantially all) of the activity of the parental nucleotide sequences from which they are derived for preventing or reducing exon 105 inclusion. In some embodiments, the AON comprises or consists of a nucleotide sequence that is selected from the group consisting of SEQ ID NOs: 42, 44, 46, 48, 51, 52, 58, 59, and 60. In some embodiments, the AON comprises or consists of a nucleotide sequence that is selected from the group consisting of SEQ ID NOs: 42, 48, and 60. In some embodiments, the AON comprises or consists of a nucleotide sequence that is selected from the group consisting of: SEQ ID NO: 24, 26, 14, 15, 16, 17, 18, 19, 20, 21, 22, 23, 25, 28, 29, 30, 31, 32, 33, 34, 35, 36, 37, 38, 39, 5, 6, 8, 9, 10, 11, 12, and 13. In a preferred embodiment, the AON of the present invention is 18, 19, 20, 21, 22, 23, 24, 25, 26, 27, or 28 nucleotides in length. More preferably, the AON according to the present invention is 24 nucleotides in length. Highly preferred AONs of the present invention consist of a nucleotide sequence according to SEQ ID NO: 24, SEQ ID NO: 26 or SEQ ID NO: 14, all being 24 nucleotides in length.

In one embodiment, the AON according to the invention comprises a region of complementarity with exon 105 of human COL7A1, wherein the region of complementarity is at most 30 nucleotides in length, preferably 8, 9, 10, 11, 12, 13, 14, 15, 16, 17, 18, 19, 20, 21, 22, 23, 24, 25, 26, 27, 28, or 29 nucleotides. A preferred region in the (pre-) mRNA of exon 105 of the human COL7A1 gene that is targeted by an AON of the present invention is the sequence 5′-TTGGCTTCATGGG-3′ (SEQ ID NO: 61). Hence, a preferred AON according to the invention comprises the sequence 5′-CCCAUGAAGCCAA-3′ (SEQ ID NO: 62).

In another aspect, the invention relates to an AON capable of preventing or reducing exon 105 inclusion into a human COL7A1 mRNA when the mRNA is produced by splicing from a pre-mRNA in a cell, wherein the AON comprises or consists of a nucleotide sequence that is complementary to a target nucleotide sequence in a COL7A1 pre-mRNA corresponding to any one of SEQ ID NOs: 48, 42, 60, 44, 46, 51, 52, 58, 59, 5-41, 43, 45, 47, 49, 50, and 53-57. In this context, a target nucleotide sequence that corresponds to a particular nucleotide sequence is the reverse complement of the particular nucleotide sequence. For example, a target nucleotide sequence in a COL7A1 pre-mRNA that corresponds to the nucleotide sequence of SEQ ID NO: 62 is the nucleotide sequence of SEQ ID NO: 61. In some embodiments, the AON comprises or consists of a nucleotide sequence that is complementary to a target nucleotide sequence in a COL7A1 pre-mRNA corresponding to any one of SEQ ID NOs: 48, 42, 60, 44, 46, 51, 52, 58, and 59. In some embodiments, the AON comprises or consists of a nucleotide sequence that is complementary to a target nucleotide sequence in a COL7A1 pre-mRNA corresponding to SEQ ID NO: 48, 42, or 60. In some embodiments, the AON comprises or consists of a nucleotide sequence that is complementary to a target nucleotide sequence in a COL7A1 pre-mRNA corresponding to any one of SEQ ID NOs: 24, 26, 14, 15, 16, 17, 18, 19, 20, 21, 22, 23, 25, 28, 29, 30, 31, 32, 33, 34, 35, 36, 37, 38, 39, 5, 6, 8, 9, 10, 11, 12, and 13. In some embodiments, the nucleotide sequence in the oligoribonucleotide that is complementary to the target nucleotide sequence has a sufficient degree of complementarity to the target nucleotide sequence such that the oligoribonucleotide can anneal to the target nucleotide sequence in a COL7A1 pre-RNA molecule under physiological conditions, thereby facilitating skipping of exon 105. In some embodiments, the nucleotide sequence in the oligoribonucleotide that is complementary to the target nucleotide sequence has a degree of complementarity to the target nucleotide sequence of about 80% or greater (such as about any of 81%, 82%, 83%, 84%, 85%, 86%, 87%, 88%, 89%, 90%, 91%, 92%, 93%, 94%, 95%, 96%, 97%, 98%, or 99%).

Preferably, the AON according to the invention is an oligoribonucleotide. More preferably, the AON comprises at least one non-natural linkage modification, even more preferably, wherein the non-natural linkage modification is a phosphorothioate linkage. The AON according to the invention, in a preferred aspect, comprises at least one nucleotide that is mono-, or disubstituted at the 2′, 3′ and/or 5′ position of the sugar moiety. Preferably, the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; O-alkyl, S-alkyl, or N-alkyl; O-alkenyl, S-alkenyl, or N-alkenyl; O-alkynyl, S-alkynyl or N-alkynyl; O-allyl, S-allyl, or N-allyl; O-alkyl-O-alkyl; -methoxy; -aminopropoxy; -methoxyethoxy; -dimethylaminooxyethoxy; and -dimethylaminoethoxyethoxy. In a highly preferred aspect, all sugar moieties of the AON according to the present invention are modified with a 2′-O-methyl (2′-OMe) substitution, or all sugar moieties are modified with a 2′-methoxyethoxy (2′-MOE) substitution. In some embodiments, all sugar moieties are modified with a 2′-OMe substitution or a 2′-MOE substitution, except for at the 5′- and 3′-terminal nucleotides, which can comprise different modifications, such as a locked nucleic acid (LNA) modification. In some embodiments, all sugar moieties are modified with a 2′-OMe substitution or a 2′-MOE substitution, except for at the two 5′- and two 3′-terminal nucleotides, which can comprise different modifications, such as an LNA modification.

In another embodiment, the invention relates to a viral vector comprising, in an expression format, a nucleotide sequence encoding an AON according to the present invention, with a sequence selected from the group consisting of SEQ ID NOs: 5-60. In some embodiments, the AON comprises or consists of a nucleotide sequence that is selected from the group consisting of SEQ ID NOs: 42, 44, 46, 48, 51, 52, 58, 59, and 60. In some embodiments, the AON comprises or consists of a nucleotide sequence that is selected from the group consisting of SEQ ID NOs: 42, 48, and 60. In some embodiments, the AON comprises or consists of a nucleotide sequence that is selected from the group consisting of: SEQ ID NO: 24, 26, 14, 15, 16, 17, 18, 19, 20, 21, 22, 23, 25, 28, 29, 30, 31, 32, 33, 34, 35, 36, 37, 38, 39, 5, 6, 8, 9, 10, 11, 12, and 13. The invention also relates to a pharmaceutical composition comprising an AON according to the invention, or a viral vector according to the invention, and further comprising one or more of a carrier, excipient, stabilizer, transfection agent, diluent, gelling agent or buffer.

In yet another aspect, the invention relates to an AON according to the invention for use in the treatment of a human subject suffering from dystrophic epidermolysis bullosa (DEB), preferably wherein the DEB is caused by a mutation in exon 105 of the COL7A1 gene in the subject, more preferably wherein the mutation is a c.7864delC mutation. Preferably, the treatment comprises topical administration of the AON, such as with a hydrogel formulation (e.g., a carbomer hydrogel formulation) comprising the AON.

In yet another aspect, the invention relates to a use of an AON according to the invention, or a viral vector according to the invention in the manufacture of a medicament for the treatment, prevention, amelioration or delay of DEB, preferably wherein the DEB is caused by a mutation in exon 105 of the COL7A1 gene, more preferably wherein the mutation is a c.7864delC mutation.

In yet another aspect, the invention relates to a method of preventing or reducing exon 105 inclusion into a human COL7A1 mRNA when the mRNA is produced by splicing from a pre-mRNA in a human cell, the method comprising providing the cell in an in vitro, in vivo or ex vivo setting, and administering to the cell an AON according to the invention, a viral vector according to the invention, or a pharmaceutical composition according to the invention. In another aspect, the invention relates to a method for the treatment of DEB in a human subject, comprising the step of administering to the subject an AON, a viral vector, or a pharmaceutical composition according to the invention. Preferably, the DEB is caused by a mutation in exon 105 of the COL7A1 gene in the human subject, more preferably wherein the mutation is a c.7864delC mutation.

In all embodiments of the present invention, the terms “preventing, or at least reducing, exon inclusion” and “exon skipping” are synonymous. In respect of COL7A1, “preventing, or at least reducing, exon inclusion” or “exon skipping” are to be construed as the exclusion of exon 105 from the human COL7A1 pre-mRNA. The term exon skipping is herein defined as the induction within a cell of a mature mRNA that does not contain a particular exon that would be present in the mature mRNA without exon skipping. Exon skipping is achieved by providing a cell expressing the pre-mRNA of the mature mRNA with a molecule capable of interfering with sequences such as, for example, the splice donor or splice acceptor sequence required for allowing the biochemical process of splicing, or with a molecule that is capable of interfering with an exon inclusion signal required for recognition of a stretch of nucleotides as an exon to be included in the mature mRNA; such molecules are also sometimes referred to as exon skipping molecules.

The term pre-mRNA refers to a non-processed or partly-processed precursor mRNA that is synthesized from a DNA template in a cell by transcription.

The term “antisense oligonucleotide” (herein generally abbreviated to AON, and sometimes elsewhere abbreviated to ASO) is understood to refer to a nucleotide sequence which is complementary to a target nucleotide sequence in a pre-mRNA molecule, hnRNA (heterogeneous nuclear RNA) or mRNA molecule, so that it is capable of annealing with its corresponding target sequence. AONs of the present invention are preferably single-stranded.

The term “complementary” as used herein includes “fully complementary” and “substantially complementary”, meaning there will usually be a degree of complementarity between the oligonucleotide and its corresponding target sequence of more than 80%, preferably more than 85%, still more preferably more than 90%, most preferably more than 95%. For example, for an oligonucleotide of 20 nucleotides in length with one mismatch between its sequence and its target sequence, the degree of complementarity is 95%. The degree of complementarity of the antisense sequence is preferably such that a molecule comprising the antisense sequence can anneal to the target nucleotide sequence in the RNA molecule under physiological conditions, thereby facilitating exon skipping. It is well known to a person having ordinary skill in the art, that certain mismatches are more permissible than others, because certain mismatches have less effect on the strength of binding, as expressed in terms of melting temperature or Tm, between AON and target sequence, than others. Certain non-complementary basepairs may form so-called “wobbles” that disrupt the overall binding to a lesser extent than true mismatches. The length of the AON also plays a role in the strength of binding; longer AONs having higher melting temperatures as a rule than shorter AONs, and the G/C content of an oligonucleotide is also a factor that determines the strength of binding, the higher the G/C content the higher the melting temperature for any given length. In the event that a single point mutation is present in exon 105 that is in the region that is complementary to the AON, it may be that the AON is not fully complementary to the target sequence (for instance when the AON is complementary to the wild type sequence) but is still effective in causing exon skipping. Certain chemical modifications of the nucleobases or the sugar-phosphate backbone, as contemplated by the present invention, may also influence the strength of binding, such that the degree of complementarity is only one factor to be taken into account when designing an oligonucleotide according to the invention.

The presence of a CpG or multitude (two or more) of CpGs in an oligonucleotide is usually associated with an increased immunogenicity of the oligonucleotide. This increased immunogenicity is undesired since it may induce damage of the tissue to be treated, i.e. the skin (dermis and/or epidermis). Thus it is preferred that an AON of the invention includes no more than 1 or 2 CpG dinucleotide sequences. More preferably, an AON of the invention includes at most 1 CpG dinucleotide sequence. Even more preferably, an AON of the invention comprises no CpG dinucleotide sequences.

The invention allows designing an oligonucleotide with acceptable RNA binding kinetics and/or thermodynamic properties. The RNA binding kinetics and/or thermodynamic properties are at least in part determined by the Tm of an oligonucleotide (calculated with the oligonucleotide properties calculator known to the person skilled in the art) for single stranded RNA using the basic Tm and the nearest neighbor models), and/or the free energy of the AON-target exon complex. If a Tm is too high, the oligonucleotide is expected to be less specific. An acceptable Tm and free energy depend on the sequence of the oligonucleotide, the chemistry of the backbone (phosphodiester, phosphorothioate, phosphoramidate, peptide-nucleic acid, etc.), the nature of the sugar moiety (ribose, deoxyribose, substituted ribose, and intra-molecular bridge) and chemical modification of the nucleobase. Therefore, the range of Tm can vary widely.

The exon skipping percentage or efficiency may be calculated by determining the concentration of wild-type band amplified, divided by the concentration of the shortened (exon 105-free) band amplified, after a given number of PCR cycles, times 100%, for any given primer set, provided the number of cycles is such that the amplification is still in the exponential phase. Quantification can be performed using the Agilent 2100 Bioanalyzer in combination with a DNA1000 kit.

Preferably, an AON according to the invention, which comprises a sequence that is complementary to a nucleotide sequence as shown in SEQ ID NO: 1 is such that the complementary part is at least about 80%, more preferably at least about 90%, still more preferably at least about 95%, most preferably about 100% complementary to the target sequence. It is thus not absolutely required that all the bases in the region of complementarity are capable of pairing with bases in the opposing strand. For instance, when designing the oligonucleotide one may want to incorporate for instance a residue that does not base pair with the base on the complementary strand. Mismatches may, to some extent, be allowed, if under the circumstances in the cell, the stretch of nucleotides is sufficiently capable of hybridizing to the complementary part. In this context, “sufficiently” means that the AONs according to the invention are capable of inducing exon skipping of exon 105. Skipping the targeted exon may conveniently be assessed by PCR/Bioanalyzer, or by digital droplet PCT (ddPCR). The complementary regions are preferably designed such that, when combined, they are specific for the exon in the pre-mRNA. Such specificity may be created with various lengths of complementary regions as this depends on the actual sequences in other (pre-) mRNA molecules in the system. The risk that the oligonucleotide also will be able to hybridize to one or more other pre-mRNA molecules decreases with increasing size of the oligonucleotide, while the length should not be too long to create problems with manufacturability, purification and/or analytics.

It is clear that AONs comprising mismatches in the region of complementarity but that retain the capacity to hybridize and/or bind to the targeted region(s) in the pre-mRNA, can be used in the present invention. However, preferably at least the complementary parts do not comprise such mismatches as these typically have a higher efficiency and a higher specificity, than AONs having such mismatches in one or more complementary regions. It is thought, that higher hybridization strengths, (i.e. increasing number of interactions with the opposing strand) are favorable in increasing the efficiency of the process of interfering with the splicing machinery of the system. Preferably, the complementarity is from 90% to 100%. In general this allows for 1 or 2 mismatches in an oligonucleotide of 20 nucleotides.

An exon skipping molecule of the invention is preferably an isolated single stranded (antisense) oligonucleotide, which is complementary to an exon 105 sequence (SEQ ID NO: 1), in which complementarity is required towards the RNA target sequence, transcribed from the human COL7A1 exon 105 DNA.

Preferably, the length of the complementary part of the oligonucleotide is the same as the length of the oligonucleotide, meaning there are no 5′ or 3′ ends of the AON that do not form a base pair with the target RNA. Thus a preferred length for an AON of the invention is 24 nucleotides or less e.g. 11, 12, 13, 14, 15, 16, 17, 18, 19, 20, 21, 22, 23, or 24. Particularly good results have been obtained with AONs having a length of 20, 21, 22, 23, or 24 nucleotides.

An AON according to the invention may contain one of more DNA residues (consequently a RNA “u” residue will be a “t” residue as DNA counterpart), or one or more RNA residues, and/or one or more nucleotide analogues or equivalents, as will be further detailed herein below.

It is preferred that an AON of the invention comprises one or more residues that are modified to increase nuclease resistance, and/or to increase the affinity of the AON for the target sequence. Therefore, in a preferred embodiment, the antisense nucleotide sequence comprises at least one nucleotide analogue or equivalent, wherein a nucleotide analogue or equivalent is defined as a residue having a (non-natural) modified base, and/or a (non-natural) modified backbone, and/or a (non-natural) internucleoside linkage, or a combination of these modifications. “Non-natural” means that the modification does not appear in nature and when such non-natural modification is introduced in an AON of the present invention (and it preferably is) it means that such AON is not a product that appears in nature, or represents a natural phenomenon.

In a preferred embodiment, the nucleotide analogue or equivalent comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells.

In one embodiment, a preferred nucleotide analogue or equivalent comprises a Peptide Nucleic Acid (PNA), having a modified polyamide backbone. PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer. Since the backbone of a PNA molecule contains no charged phosphate groups, PNA-RNA hybrids are usually more stable than RNA-RNA or RNA-DNA hybrids, respectively.

According to one embodiment of the invention the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

In yet another embodiment, a nucleotide analogue or equivalent of the invention comprises a substitution of one of the non-bridging oxygens in the phosphodiester linkage. This modification slightly destabilizes base-pairing but adds significant resistance to nuclease degradation. A preferred nucleotide analogue or equivalent comprises a phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. It should be understood, that the invention preferably encompasses an AON that can bind to a target nucleic acid, wherein at least one internucleosidic linkage comprises a chiral center (including X-phosphonate moieties, wherein X may be alkyl, alkoxy, aryl, alkylthio, acyl, —NR¹R¹, alkenyloxy, alkynyloxy, alkenylthio, alkynylthio, —S—Z⁺, —Se—Z⁺, or —BH₃—Z⁺, and wherein R¹ is independently hydrogen, alkyl, alkenyl, alkynyl, or aryl, and wherein Z⁺ is ammonium ion, alkylammonium ion, heteroaromatic iminium ion, or heterocyclic iminium ion, any of which is primary, secondary, tertiary or quaternary, or Z is a monovalent metal ion. Both the determination of the tolerability of such linkages per se, using computational modelling, as well as the determination of the preferred Sp or Rp stereomer of that linkage comprising a chiral centre forms part of the invention.

The invention, in one preferred embodiment, relates to an AON, wherein the intemucleosidic linkage that displays chirality is a phosphorotioate linkage. In a further preferred embodiment, the AON of the present invention comprises at least one intemucleotide linkage with a predetermined Rp or Sp phosphorothioate configuration, which means that during the manufacturing of the AON an Rp or Sp configuration of the phosphorothioate linkage is selected to improve the AON's stability and or efficiency towards the target sequence. Further to that, the AON may be completely stereopure in the sense that all phosphorothioate linkages in the AON have an Rp or Sp configuration (or combinations thereof throughout the AON) that is pre-selected.

A further preferred nucleotide analogue or equivalent of the invention comprises one or more sugar moieties that are mono- or di-substituted at the 2′, 3′ and/or 5′ position such as:
—OH; —F;
substituted or unsubstituted, linear or branched lower (C1-C10) alkyl, alkenyl, alkynyl, alkaryl, allyl, or aralkyl, that may be interrupted by one or more heteroatoms;
O-, S-, or N-alkyl;
O-, S-, or N-alkenyl;
O-, S- or N-alkynyl;
O-, S-, or N-allyl;
O-alkyl-O-alkyl;
-methoxy;
-aminopropoxy;
-methoxyethoxy;
-dimethylaminooxyethoxy; or
-dimethylaminoethoxyethoxy.

Especially preferred modifications of the sugar moiety are 2′-O-methyl (2′-OMe) and 2′-methoxyethoxy (2′-O-methoxyethyl, or 2′-MOE) modifications, as further outlined in the non-limiting examples disclosed herein. The sugar moiety can also be a furanose or derivative thereof, or a deoxyfuranose or derivative thereof, preferably ribose or derivative thereof, or deoxyribose or derivative of A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-0,4′-C-ethylene-bridged nucleic acid. These substitutions render the nucleotide analogue or equivalent RNaseH and nuclease resistant and increase the affinity for the target RNA. In some embodiments, an AON of the invention comprises more than one type of sugar moiety modification. For example, in some embodiments of an AON of the invention the 5′- and 3′-terminal nucleotides are LNA-modified, and all other sugar moieties are modified with a 2′-OMe substitution or a 2′-MOE substitution, and in other embodiments the two 5′- and two 3′-terminal nucleotides are LNA-modified, and all other sugar moieties are modified with a 2′-OMe substitution or a 2′-MOE substitution.

It is understood by a skilled person that it is not necessary for all internucleosidic linkages in an antisense oligonucleotide to be modified. For example, some internucleosidic linkages may be unmodified, whereas other internucleosidic linkages are modified. AONs comprising a backbone consisting of one form of (modified) internucleosidic linkages, multiple forms of (modified) internucleosidic linkages, uniformly or non-uniformly distributed along the length of the AON are all encompassed by the present invention. In addition, any modality of backbone modification (uniform, non-uniform, mono-form or pluriform and all permutations thereof) may be combined with any form or of sugar or nucleoside modifications or analogues mentioned below.

An especially preferred backbone for the AONs according to the invention is a uniform (all) phosphorothioate (PS) backbone, which may be (as outlined above) made stereopure in chirality (with pre-selected Rp and/or Sp configurations).

In another embodiment, a nucleotide analogue or equivalent of the invention comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

It is understood by a skilled person that it is not necessary for all positions in an AON to be modified uniformly. In addition, more than one of the aforementioned analogues or equivalents may be incorporated in a single AON or even at a single position within an AON. In certain embodiments, an AON of the invention has at least two different types of analogues or equivalents.

According to a preferred embodiment AONs according to the invention comprise a 2′-O (preferably lower) alkyl phosphorothioate AON, such as 2′-O-methyl (2′-OMe) modified ribose (RNA), 2′-O-methoxyethyl (=2′-methoxyethoxy; 2′-MOE) modified ribose, 2′-O-ethyl modified ribose, 2′-O-propyl modified ribose, and/or substituted derivatives of these modifications such as halogenated derivatives.

An effective and preferred AON format according to the invention comprises 2′-O-methyl (2′-OMe) modified ribose moieties with a phosphorothioate backbone, preferably wherein substantially all ribose moieties are 2′-OMe and substantially all internucleosidic linkages are phosphorothioate linkages. Yet another effective and preferred AON format according to the invention comprises 2′-O-methoxyethyl (2′-MOE) modified ribose moieties with a phosphorothioate backbone, preferably wherein substantially all ribose moieties are 2′-MOE and substantially all internucleosidic linkages are phosphorothioate linkages.

In some embodiments, provided herein is an AON according to the invention that comprises or consists of a nucleotide sequence that is selected from the group consisting of SEQ ID NOs: 5-60, wherein all sugar moieties are modified with a 2′-O-methyl (2′-OMe) substitution. In some embodiments, the AON comprises or consists of a nucleotide sequence that is selected from the group consisting of SEQ ID NOs: 48, 42, 60, 44, 46, 51, 52, 58, and 59. In some embodiments, the AON comprises or consists of a nucleotide sequence that is selected from the group consisting of SEQ ID NOs: 48, 42, and 60. In some embodiments, the AON comprises or consists of the nucleotide sequence of SEQ ID NO: 48. In some embodiments, the AON comprises or consists of the nucleotide sequence of SEQ ID NO: 42. In some embodiments, the AON comprises or consists of the nucleotide sequence of SEQ ID NO: 60. In some embodiments, the AON comprises or consists of a nucleotide sequence that is selected from the group consisting of SEQ ID NOs: 24, 26, 14, 15, 16, 17, 18, 19, 20, 21, 22, 23, 25, 28, 29, 30, 31, 32, 33, 34, 35, 36, 37, 38, 39, 5, 6, 8, 9, 10, 11, 12, and 13. In some embodiments, the AON has a full phosphorothioate backbone.

In some embodiments, provided herein is an AON according to the invention that comprises or consists of a nucleotide sequence that is selected from the group consisting of SEQ ID NOs: 5-60, wherein all sugar moieties are modified with a 2′-methoxyethoxy (2′-MOE) substitution. In some embodiments, the AON comprises or consists of a nucleotide sequence that is selected from the group consisting of SEQ ID NOs: 48, 42, 60, 44, 46, 51, 52, 58, and 59. In some embodiments, the AON comprises or consists of a nucleotide sequence that is selected from the group consisting of SEQ ID NOs: 48, 42, and 60. In some embodiments, the AON comprises or consists of the nucleotide sequence of SEQ ID NO: 48. In some embodiments, the AON comprises or consists of the nucleotide sequence of SEQ ID NO: 42. In some embodiments, the AON comprises or consists of the nucleotide sequence of SEQ ID NO: 60. In some embodiments, the AON comprises or consists of a nucleotide sequence that is selected from the group consisting of SEQ ID NOs: 24, 26, 14, 15, 16, 17, 18, 19, 20, 21, 22, 23, 25, 28, 29, 30, 31, 32, 33, 34, 35, 36, 37, 38, 39, 5, 6, 8, 9, 10, 11, 12, and 13. In some embodiments, the AON has a full phosphorothioate backbone.

In some embodiments, provided herein is an AON according to the invention that comprises or consists of a nucleotide sequence that is selected from the group consisting of SEQ ID NOs: 5-60, wherein the 5′- and 3′-terminal nucleotides are LNA-modified, and all other sugar moieties are modified with a 2′-OMe substitution or a 2′-MOE substitution. In some embodiments, all the other sugar moieties are modified with a 2′-OMe substitution. In some embodiments, all the other sugar moieties are modified with a 2′-MOE substitution. In some embodiments, the AON comprises or consists of a nucleotide sequence that is selected from the group consisting of SEQ ID NOs: 48, 42, 60, 44, 46, 51, 52, 58, and 59. In some embodiments, the AON comprises or consists of a nucleotide sequence that is selected from the group consisting of SEQ ID NOs: 48, 42, and 60. In some embodiments, the AON comprises or consists of the nucleotide sequence of SEQ ID NO: 48. In some embodiments, the AON comprises or consists of the nucleotide sequence of SEQ ID NO: 42. In some embodiments, the AON comprises or consists of the nucleotide sequence of SEQ ID NO: 60. In some embodiments, the AON comprises or consists of a nucleotide sequence that is selected from the group consisting of SEQ ID NOs: 24, 26, 14, 15, 16, 17, 18, 19, 20, 21, 22, 23, 25, 28, 29, 30, 31, 32, 33, 34, 35, 36, 37, 38, 39, 5, 6, 8, 9, 10, 11, 12, and 13. In some embodiments, the AON has a full phosphorothioate backbone.

In some embodiments, provided herein is an AON according to the invention that comprises or consists of a nucleotide sequence that is selected from the group consisting of SEQ ID NOs: 5-60, wherein the two 5′- and two 3′-terminal nucleotides are LNA-modified, and all other sugar moieties are modified with a 2′-OMe substitution or a 2′-MOE substitution. In some embodiments, all the other sugar moieties are modified with a 2′-OMe substitution. In some embodiments, all the other sugar moieties are modified with a 2′-MOE substitution. In some embodiments, the AON comprises or consists of a nucleotide sequence that is selected from the group consisting of SEQ ID NOs: 48, 42, 60, 44, 46, 51, 52, 58, and 59. In some embodiments, the AON comprises or consists of a nucleotide sequence that is selected from the group consisting of SEQ ID NOs: 48, 42, and 60. In some embodiments, the AON comprises or consists of the nucleotide sequence of SEQ ID NO: 48. In some embodiments, the AON comprises or consists of the nucleotide sequence of SEQ ID NO: 42. In some embodiments, the AON comprises or consists of the nucleotide sequence of SEQ ID NO: 60. In some embodiments, the AON comprises or consists of a nucleotide sequence that is selected from the group consisting of SEQ ID NOs: 24, 26, 14, 15, 16, 17, 18, 19, 20, 21, 22, 23, 25, 28, 29, 30, 31, 32, 33, 34, 35, 36, 37, 38, 39, 5, 6, 8, 9, 10, 11, 12, and 13. In some embodiments, the AON has a full phosphorothioate backbone.

It will also be understood by a skilled person that different AONs can be combined for efficient skipping of exon 105 of the COL7A1 gene. A combination of two or more different AONs may be used in a method of the invention, such as two different AONs, three different AONs, four different AONs, or five different AONs targeting the same or different regions of exon 105, as long as at least one AON is one according to the invention. In some embodiments, the combination of two or more different AONs includes a first AON of the invention that comprises or consists of the nucleotide sequence of SEQ ID NO: 42 and a second AON of the invention that comprises or consists of the nucleotide sequence of SEQ ID NO: 44. In some embodiments, the combination of two or more different AONs includes a first AON of the invention that comprises or consists of the nucleotide sequence of SEQ ID NO: 45 and a second AON of the invention that comprises or consists of the nucleotide sequence of SEQ ID NO: 52. In some embodiments, the combination of two or more different AONs includes a first AON of the invention that comprises or consists of the nucleotide sequence of SEQ ID NO: 48 and a second AON of the invention that comprises or consists of the nucleotide sequence of SEQ ID NO: 51.

An AON can be linked to a moiety that enhances uptake of the AON in cells, preferably skin cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a camelid single domain antigen-binding domain. A preferred administration method makes use of a hydrogel in which the AON of the invention is formulated, such as a carbomer hydrogel.

An AON according to the invention may be a naked (gymnotic) AON or in the form of a conjugate or expressed from a vector (vectored AON). The AON may be administrated using suitable means known in the art. When the exon skipping molecule is a vectored AON, it may for example be provided to an individual or a cell, tissue or organ of the individual in the form of an expression vector wherein the expression vector encodes a transcript comprising the oligonucleotide. The expression vector is preferably introduced into a cell, tissue, organ or individual via a gene delivery vehicle, such as a viral vector. In a preferred embodiment, there is provided a viral-based expression vector comprising an expression cassette or a transcription cassette that drives expression or transcription of an exon skipping molecule as identified herein. Accordingly, the present invention provides a viral vector expressing an AON according to the invention when placed under conditions conducive to expression of the exon skipping molecule. A cell can be provided with an exon skipping molecule capable of interfering with sequences essential for, or at least conducive to, exon 105 inclusion, such that such interference prevents, or at least reduces, exon 105 inclusion into the COL7A1 mRNA, for example by plasmid-derived AON expression or viral expression provided by adenovirus- or adeno-associated virus-based vectors. Expression may be driven by a polymerase III promoter, such as a U1, a U6, or a U7 RNA promoter. A preferred delivery vehicle is a viral vector such as an adeno-associated virus vector (AAV), or a retroviral vector such as a lentivirus vector and the like. Also, plasmids, artificial chromosomes, plasmids usable for targeted homologous recombination and integration in the mammalian (preferably human) genome of cells may be suitably applied for delivery of an oligonucleotide as defined herein. Preferred for the current invention are those vectors wherein transcription is driven from Pol-III promoters, and/or wherein transcripts are in the form of fusions with U1 or U7 transcripts, which yield good results for delivering small transcripts. It is within the skill of the artisan to design suitable transcripts. Preferred are Pol-III driven transcripts. Preferably, in the form of a fusion transcript with an U1 or U7 transcript.

The invention also provides a viral-based vector, comprising a Pol III-promoter driven expression cassette for expression of an AON of the invention for inducing skipping of COL7A1 exon 105. An AAV vector according to the present invention is a recombinant AAV vector and refers to an AAV vector comprising part of an AAV genome comprising an encoded AON according to the invention encapsidated in a protein shell of capsid protein derived from an AAV serotype as depicted elsewhere herein. Part of an AAV genome may contain the inverted terminal repeats (ITR) derived from an adeno-associated virus serotype, such as AAV1, AAV2, AAV3, AAV4, AAV5, AAV6, AAV7, AAV8, AAV9 and others. Protein shell comprised of capsid protein may be derived from an AAV serotype such as AAV1, 2, 3, 4, 5, 6, 7, 8, 9 and others. A protein shell may also be named a capsid protein shell. AAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the present invention a capsid protein shell may be of a different serotype than the AAV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV5 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention. Preferably, a recombinant AAV vector according to the present invention comprises a capsid protein shell of AAV serotype 2, 5, 6, 7, 8 or AAV serotype 9 wherein the AAV genome or ITRs present in the AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector, respectively. More preferably, a recombinant AAV vector according to the present invention has tropism for dermal and epidermal cells and comprises a capsid protein shell of AAV serotype 5 or 8. The AAV genome or ITRs present in the vector may be derived from the same or a different serotype, such as AAV serotype 2; such vector is referred to as an AAV 2/5 or AAV 2/8 vector. AAV with a serotype 5 capsid have tropism for dermal and epidermal cells, such as basal and suprabasal keratinocytes and dermal fibroblasts. AAV vectors with a type 5 capsid display much higher transduction efficiencies compared to AAV with a type 2 capsid. Similarly, AAV with a capsid of serotype 8 show tropism towards dermal fibroblasts and (mainly) suprabasal keratinocytes. Moreover, AAV 2/8 tend to be more efficient in transducing mammalian, preferably human dermal and epidermal cells than AAV 2/5. However, transduction efficiency appears to depend on the timing of administration during wound healing, AAV 2/2 showing higher transduction efficiencies than AAV 2/5 and AAV 2/8 at later time points. Hence, AAV 2/2, AAV x/5 and AAV x/8 are preferred AAV to deliver AONs according to the invention and their choice may be determined taking into account the time of administration and the cell types to be targeted. These details can be readily worked out a person skilled in the art, in pre-clinical or clinical studies. A nucleic acid molecule encoding an AON according to the present invention represented by a nucleic acid sequence of choice is preferably inserted between the AAV genome or ITR sequences as identified above, for example an expression construct comprising an expression regulatory element operably linked to a coding sequence and a 3′ termination sequence.

“AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art. The AAV helper functions can be supplied on an AAV helper construct, which may be a plasmid. Introduction of the helper construct into the host cell can occur e.g. by transformation, transfection, or transduction prior to or concurrently with the introduction of the AAV genome present in the AAV vector as identified herein. The AAV helper constructs of the invention may thus be chosen such that they produce the desired combination of serotypes for the AAV vector's capsid protein shell on the one hand and for the AAV genome present in the AAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456. Preferably, an AAV genome as present in a recombinant AAV vector according to the present invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

Improvements in means for providing an individual or a cell, tissue, organ of the individual with an exon skipping molecule according to the invention, are anticipated considering the progress that has already thus far been achieved. Such future improvements may of course be incorporated to achieve the mentioned effect on restructuring of mRNA using a method of the invention. An AON according to the invention can be delivered as is to an individual, a cell, tissue or organ of the individual. When administering an AON according to the invention, it is preferred that the molecule is dissolved in a solution that is compatible with the delivery method.

Gymnotic AONs are readily taken up by most cells in vivo, and usually dissolving the AONs according to the invention in an isotonic (saline) solution will be sufficient to reach the target cells, such as skin (dermis and epidermis) cells. Alternatively, gymnotic AONs of the invention may be formulated using pharmaceutically acceptable excipients, additives, stabilizers, solvents, colorants and the like. In addition, or alternatively, gymnotic AONs may be formulated with any of the transfection aids mentioned below.

Skin (dermis and epidermis) cells can be provided with a plasmid for AON expression by providing the plasmid in an aqueous solution, such as an isotonic (saline) solution. Alternatively, a plasmid can be provided by transfection using known transfection agents.

For intravenous, subcutaneous, intramuscular, intrathecal and/or intraventricular administration it is preferred that the solution is an isotonic (saline) solution. Particularly preferred in the invention is the use of an excipient or transfection agents that will aid in delivery of each of the constituents as defined herein to a cell and/or into a cell, preferably a skin (dermis and epidermis) cell. Preferred are excipients or transfection agents capable of forming complexes, nanoparticles, micelles, vesicles and/or liposomes that deliver each constituent as defined herein, complexed or trapped in a vesicle or liposome through a cell membrane. Many of these excipients are known in the art. Suitable excipients or transfection agents comprise polyethylenimine (PEI; ExGen500 (MBI Fermentas)), LipofectAMINE™ 2000 (Invitrogen) or derivatives thereof, or similar cationic polymers, including polypropyleneimine or polyethylenimine copolymers (PECs) and derivatives, synthetic amphiphils (SAINT-18), Lipofectin™, DOTAP and/or viral capsid proteins that are capable of self-assembly into particles that can deliver each constituent as defined herein to a cell, preferably a skin (dermis r epidermis) cell. Such excipients have been shown to efficiently deliver an oligonucleotide such as AONs to a wide variety of cultured cells, including skin (dermis and epidermis) cells. Their high transfection potential is combined with an acceptably low to moderate toxicity in terms of overall cell survival. The ease of structural modification can be used to allow further modifications and the analysis of their further (in vivo) nucleic acid transfer characteristics and toxicity. Lipofectin represents an example of a liposomal transfection agent. It consists of two lipid components, a cationic lipid N-[1-(2,3 dioleoyloxy)propyl]-N,N,N-trimethylammonium chloride (DOTMA) (cp. DOTAP which is the methylsulfate salt) and a neutral lipid dioleoylphosphatidylethanolamine (DOPE). The neutral component mediates the intracellular release. Another group of delivery systems are polymeric nanoparticles. Polycations such like diethylaminoethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver each constituent as defined herein, preferably an oligonucleotide, across cell membranes into cells.

In addition to these common nanoparticle materials, the cationic peptide protamine offers an alternative approach to formulate an oligonucleotide with colloids. This colloidal nanoparticle system can form so called proticles, which can be prepared by a simple self-assembly process to package and mediate intracellular release of an oligonucleotide. The skilled person may select and adapt any of the above or other commercially available alternative excipients and delivery systems to package and deliver an exon skipping molecule for use in the current invention to deliver it for the prevention, treatment or delay of a disease or condition associated with a mutated exon 105 in the COL7A1 gene.

An AON according to the invention could be covalently or non-covalently linked to a targeting ligand specifically designed to facilitate the uptake into the cell (especially a skin (dermis) cell), cytoplasm and/or its nucleus. Such ligand could comprise (i) a compound (including but not limited to peptide(-like) structures) recognizing cell, tissue or organ specific elements facilitating cellular uptake and/or (ii) a chemical compound able to facilitate the uptake in to cells and/or the intracellular release of an oligonucleotide from vesicles, e.g. endosomes or lysosomes.

Therefore, in a preferred embodiment, an AON according to the invention is formulated in a composition or a medicament or a composition, which is provided with at least an excipient and/or a targeting ligand for delivery and/or a delivery device thereof to a cell and/or enhancing its intracellular delivery.

It is to be understood that if a composition comprises an additional constituent such as an adjunct compound as later defined herein, each constituent of the composition may be formulated in one single combination or composition or preparation. Depending on their identity, the skilled person will know which type of formulation is the most appropriate for each constituent as defined herein. According to one embodiment, the invention provides a composition or a preparation which is in the form of a kit of parts comprising an AON according to the invention and a further adjunct compound as later defined herein.

If required, an AON according to the invention or a vector, preferably a viral vector, expressing an AON according to the invention can be incorporated into a pharmaceutically active mixture by adding a pharmaceutically acceptable carrier.

Accordingly, the invention also provides a composition, preferably a pharmaceutical composition, comprising an AON according to the invention, such as gymnotic AON or a viral vector according to the invention and a pharmaceutically acceptable excipient. Such composition may comprise a single AON according to the invention, but may also comprise multiple, distinct AONs according to the invention. Such a pharmaceutical composition may comprise any pharmaceutically acceptable excipient, including a carrier, excipient, stabilizer, transfection agent, gelling agent, buffer, filler, preservative, adjuvant, solubilizer and/or diluent. Such pharmaceutically acceptable components may for instance be found in Remington, 2000. Each feature of the composition has earlier been defined herein.

If multiple distinct AONs according to the invention are used, concentration or dose defined herein may refer to the total concentration or dose of all oligonucleotides used or the concentration or dose of each AON used or added. Therefore in one embodiment, there is provided a composition wherein each or the total amount of AONs according to the invention used is dosed in an amount ranged from 0.0001 and 100 mg/kg, preferably from 0.001 and 50 mg/kg, still more preferably between 0.01 and 20 mg/kg.

A preferred AON according to the invention is for the treatment of DEB or, more generally, a mutated COL7A1 exon 105 related disease or condition of an individual. In all embodiments of the present invention, the term “treatment” is understood to include the prevention and/or delay of the disease or condition. An individual, which may be treated using an AON according to the invention may already have been diagnosed as having DEB or a COL7A1 exon 105 related disease or condition. Alternatively, an individual which may be treated using an AON according to the invention may not have yet been diagnosed, but may be an individual having an increased risk of developing DEB, or a COL7A1 exon 105 related disease or condition in the future given his or her genetic background.

One preferred method of administration of AONs according to the invention is by the appliance of AON-coated bandages capable of releasing the AONs. Especially beneficial are multilayered (Layer-by-Layer, LbL)-coated bandages such as disclosed in WO2014/150074. It discloses prolonged and effective release of a wound-healing-promoting siRNA from an adhesive bandage, coated with a multi-layered film containing the siRNA. A bandage that may suitably be used in combination with AONs according to the invention, is Tegaderm®. Suitable multilayer coatings for the delivery of siRNA that may also be used in combination with AONs according to the invention, comprises a Laponite® containing layer-by-layer architecture. Other bandages than Tegaderm® that are capable of releasing nucleic acid therapeutics, may be used. Also non-adhesive bandages may be used, as they are likely to be less painful for the patient, as long as the bandage is in close contact with the skin or the wound-site. AON-containing LBL films for delivery of AONs according to the invention in combination with bandages are described in WO2014/150074. Dosing may be daily, weekly, monthly, quarterly, once per year, depending on the route of administration and the need of the patient.

Because of the early onset of disease, patients having or at risk of developing a disease, disorder or condition caused by or associated with a mutated exon 105 of the COL7A1 gene, including DEB, may be treated in utero, directly after birth, from 1, 2, 3, 6 months of age, from one year of age, from 3 years of age, from 5 years of age, prior to or after the onset of symptoms, to alleviate, retard development, stop or reverse the symptoms of disease and the like.

A treatment in a use or in a method according to the invention is at least one week, at least one month, at least several months, at least one year, at least 2, 3, 4, 5, 6 years or chronically, even during a patient's entire life. Each exon skipping molecule or AON or equivalent thereof as defined herein for use according to the invention may be suitable for direct administration to a cell, tissue and/or an organ in vivo of individuals already affected or at risk of developing a mutated COL7A1 exon 105 related disorder, disease or condition, and may be administered directly in vivo, ex vivo or in vitro. The frequency of administration of an AON, composition, compound or adjunct compound of the invention may depend on several parameters such as the age of the patient, the nature of the exon skipping molecule (e.g. gymnotic AON or vectored AON, such as AAV or lentiviral vector expressed AONs), the dose, the formulation of the molecule and the like.

Dose ranges of an exon skipping molecule, preferably an AON according to the invention are preferably designed on the basis of rising dose studies in clinical trials (in vivo use) for which rigorous protocol requirements exist. An AON as defined herein may be used at a dose range from 0.0001 to 100 mg/kg, preferably from 0.01 to 20 mg/kg. The dose and treatment regime may vary widely, depending on many factors, including but not limited to the route of administration (e.g. systemic versus topically), whether the AON is administered as a gymnotic AON or as vectored AON, the dosing regimen, the age and weight of the patient, and so forth.

In a preferred embodiment, a viral vector, preferably an AAV vector as described earlier herein, as delivery vehicle for an AON according to the invention, is administered in a dose ranging from 1×10⁹-1×10¹⁷ virus particles per injection, more preferably from 1×10¹⁰-1×10¹⁴, and most preferably 1×10¹⁰-1×10¹² virus particles per injection.

It will be clear to a person having ordinary skill in the art to which this invention pertains, that the details of treatment will need to be established in accordance with and depending on such factors as the sequence and chemistry of the oligonucleotide(s), the route of administration, the formulation, the dose, the dosing regimen, the format (viral vector or gymnotic oligonucleotide), the age and weight of the patient, the stage of the disease and so forth, which may require further non-clinical and clinical investigation.

Unless otherwise indicated each embodiment as described herein may be combined with another embodiment as described herein.

As can be observed in the experimental section and the examples herein, at the RNA level, addition of various AONs according to the invention targeting exon 105 of the COL7A1 gene indeed resulted in a mRNA lacking exon 105, leading to the production of a shorter but functional collagen VII protein.

In fibroblasts (that can be derived from skin cells), collagen VII is abundantly expressed. Therefore, it is to be expected that addition of AONs to cultured fibroblasts from DEB patients will result in an increased amount of shortened but functional collagen VII protein that is detectable on Western blot, and as such will demonstrate that AON-based therapy will not only redirect splicing of the COL7A1 mRNA but will also result in restoring collagen VII functionality.

The terms “adenine”, “guanine”, “cytosine”, “thymine”, “uracil” and hypoxanthine (the nucleobase in inosine) refer to the nucleobases as such. The terms adenosine, guanosine, cytidine, thymidine, uridine and inosine, refer to the nucleobases linked to the (deoxy)ribosyl sugar. The term “nucleoside” refers to the nucleobase linked to the (deoxy)ribosyl sugar.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “include” and all of its tenses and conjugations, is to be read as “include, but is not limited to”.

The word “exon skipping molecule” is meant to include gymnotic AONs and vectored AONs, including viral vectors, capable of expressing AONs in a compatible cell.

The word “about” or “approximately” when used in association with a numerical value (e.g. about 10) preferably means that the value may be the given value (of 10) plus or minus 5% of the value.

### EXAMPLES

### Example 1: Design and Use of AONs for Exon 105 Exclusion from Human COL7A1 Pre-mRNA

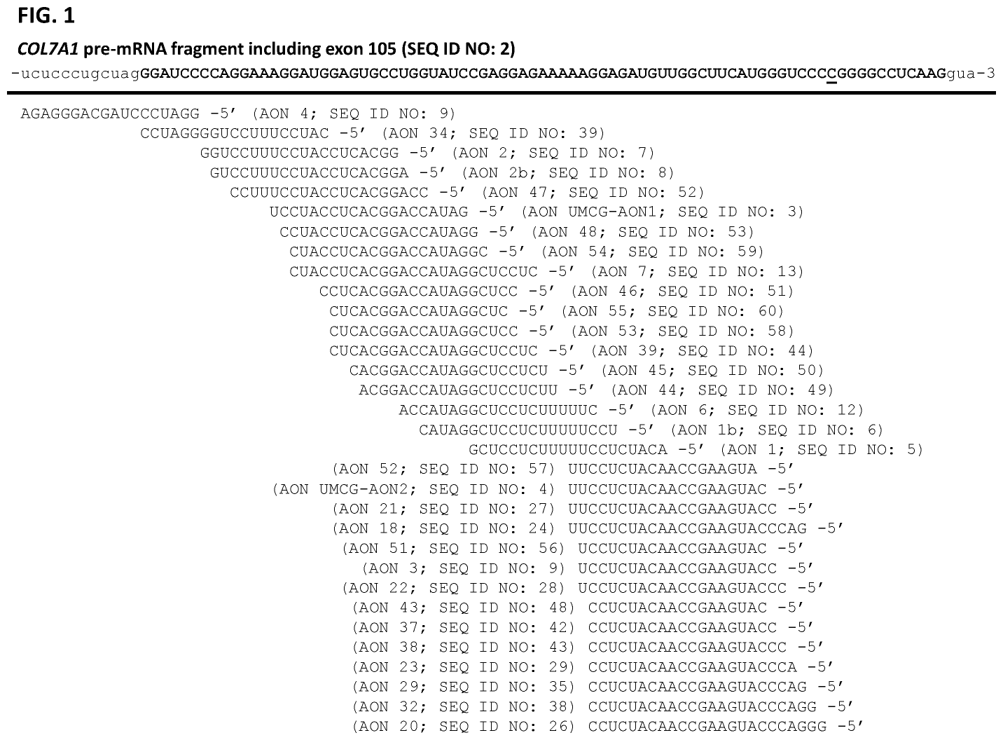

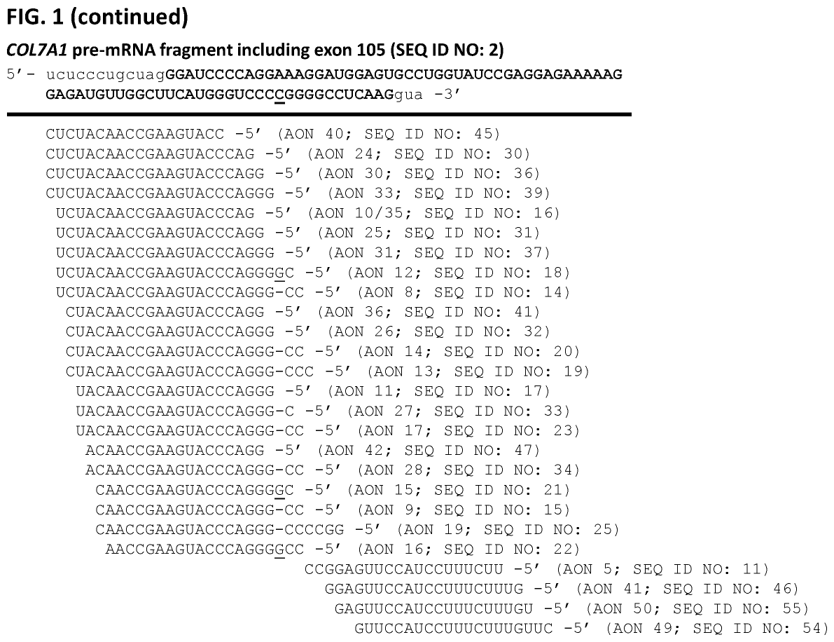

To establish exon 105 skipping from human COL7A1 pre-mRNA the inventors of the present invention designed 31 new AONs that were assessed for activity in comparison to two AONs previously disclosed in Bremer et al. (2016) and WO2017/078526. Two chemical modifications were specifically addressed as well: AONs that were fully modified with 2′-OMe at the sugar moiety or that were fully modified with 2′-MOE at the sugar moiety. All AONs were modified with phosphorothioate linkages connecting the nucleosides. Table 1 shows the AONs that were assessed, with their SEQ ID NOs and in what 2′ substituted versions they were tested. FIG. 1 shows the respective positions of all these AONs in relation to their complementary sequence in exon 105 (top). Underlined in FIG. 1 is the C position that is deleted in the c.7864delC mutation known from the art (Escámez et al. 2010). AONs 12, 15, and 16 are 100% complementary to the exon 105 sequence and overlap with the wild type sequence. AONs 8, 9, 13, 14, 17, 19, 27, and 28 are 100% complementary to the c.7864delC mutant sequence and overlap with the c.7864delC mutation (thereby lacking the opposing G). The other AONs are outside this mutation and 100% complementary to the wild type sequence.

TABLE 1
AONs tested for skipping of exon 105. AONs were tested with
different chemical modifications as indicated by an “x” in the
respective column.

| | | Modifications tested | |
|---|---|---|---|
| AON | SEQ ID NO | MOE + PS¹ | OMe + PS² |
| AON4 | 9 | x | |
| AON2 | 7 | | |
| AON2b | 8 | x | |
| UMCG-AON1 | 3 | x | x |
| AON7 | 13 | x | x |
| AON6 | 12 | x | x |
| AON1b | 6 | x | |
| AON1 | 5 | x | x |
| UMCG-AON2 | 4 | x | x |
| AON21 | 27 | | x |
| AON18 | 24 | x | x |
| AON3 | 9 | x | |
| AON22 | 28 | | x |
| AON23 | 29 | | x |
| AON29 | 35 | | x |
| AON32 | 38 | | x |
| AON20 | 26 | x | x |
| AON24 | 30 | | x |
| AON30 | 36 | | x |
| AON33 | 39 | | x |
| AON10 | 16 | x | x |
| AON25 | 31 | | x |
| AON31 | 37 | | x |
| AON12 | 18 | x | x |
| AON8 | 14 | x | x |
| AON26 | 32 | | x |
| AON14 | 20 | x | |
| AON13 | 19 | x | |
| AON11 | 17 | x | x |
| AON27 | 33 | | x |
| AON17 | 23 | x | |
| AON28 | 34 | | x |
| AON15 | 21 | x | |
| AON9 | 15 | x | x |
| AON19 | 25 | x | |
| AON16 | 22 | x | |
| AON5 | 11 | x | |

¹MOE + PS: Fully 2′-MOE-modified with PS backbone.
²OMe + PS: Fully 2′-OMe-modified with PS backbone.

In a first experiment, the two UMCG AONs (UMCG-AON1, SEQ ID NO: 3; and UMCG-AON2, SEQ ID NO: 4) known from Bremer et al. (2016) and WO2017/078526 (both in a 2′-OMe and a 2′-MOE version) were compared to AON8 in a 2′-MOE version generated by the inventors of the present invention.

For this, wild type fibroblasts were cultured and transfected with the designed AONs as follows. First fibroblasts were grown to 80-90% confluency, washed with PBS and trypsinized. The cells were counted using an EVE cell counter (NanoEntek) and checked for viability (>80% viability prerequisite). The cells were seeded at 1.5×10⁵ cells/well in a 6 well plate. Plates were incubated overnight and the next day the cells were transfected with 250 nM of the different AONs, including the UMCG-AON1 and UMCG-AON2 and a combination of these 2 (total end concentration 250 nM), using Max PEI as transfection reagent (Polysciences). The charge ratio PEI:AON was 1.8 μL:400 ng for transfections using 100 nM, and 1:2 ratio in weight (AON;Pei) for transfections with 250 nM. After 24 h the samples were harvested for RNA isolation. For this, cells were washed with PBS and lysed with 250 μL BL+TG lysis buffer of the ReliaPrep™ RNA Cell Miniprep System. mRNA was isolated according to the manufacturers protocol. The RNA was eluted in 25 μL RNAse-free water and the concentrations and purities were measured using OD ratio's 260/280 and 260/230 with the Nanodrop 2000 (Thermo Fisher). The cDNA synthesis was performed using 300 ng RNA. The reaction mix was added according to the manufacturer's protocol (Verso Kit, Thermo Fisher). cDNA synthesis was performed for 30 min at 42° C. Random Hexamer primer (provided by the Verso kit) was used to synthesize the first strand cDNA. Subsequently, PCRs were performed with the Mastermix (kit and dNTPs both from Applied Biosystems by Thermo Fisher Scientific) with 1 μl of cDNA template using a FW primer (5′-GTGACAAAGGACCTCGGGG-3′ SEQ ID NO: 63), and a Reverse primer (5′-CTCCATCAAGGCCACAGGC-3′ SEQ ID NO: 64). The PCR was run at 62° C. for 35 cycles. PCR products were analysed using lab on a chips technology (the DNA1000 kit, Bioanalyzer, Agilent) which detects different fragment sizes and performs quantitative analysis based on yield. The DNA 1000 chip was used. Visualization was performed using the Agilent Bioanalyzer 2100.

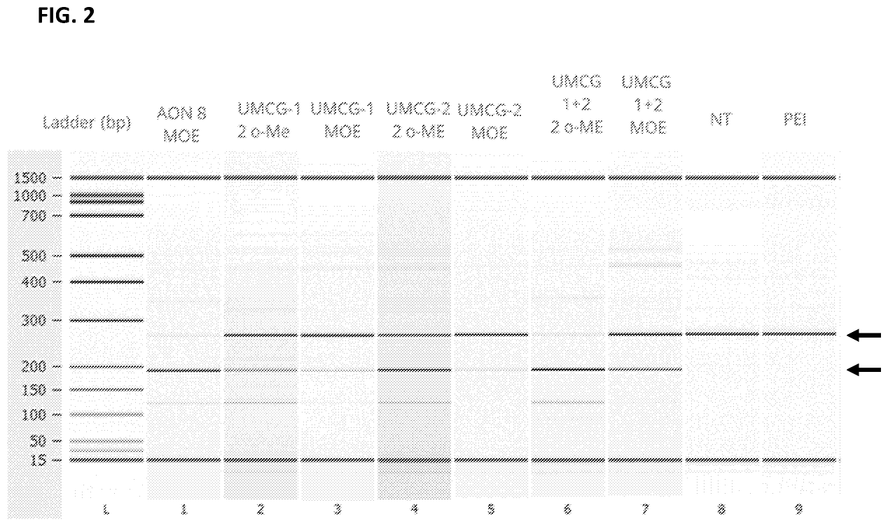

FIG. 2 shows the results of the PCR on a Bioanalyzer. AON8 (2′-MOE version) showed significant skipping of exon 105, especially in comparison to the 2′-MOE versions of the oligonucleotides UMCG-1 and UMCG-2 AONs known from the art. In line with what was shown in Bremer et al. (2016) the single use of AON1 (2′-MOE) and AON2 (2′-MOE) did not give any detectable skip, whereas exon 105 skipping was observed when the AONs were used in combination. The same effect was seen when the 2′-OMe versions of the UMCG AONs were used alone or in combination, although the effect with these the 2′-OMe version appeared more prominent than with the 2′-MOE versions. AON8 (2′-MOE) outperformed both UMCG-1 (2′-MOE) and UMCG-2 (2′-MOE) compounds, when used alone. As outlined herein above, it is preferred for development and therapeutic purposes (manufacturing, toxicity, tissue entry, etc.) to have a single active compound to achieve exon 105 skipping. From this initial experiment, it was concluded that AON8 has superior skipping efficiency in comparison to the two AONs from the art that were earlier shown to yield exon 105 skipping, when they were combined. It should be noted that AON8 is 100% complementary with the c.7864delC mutant mRNA (by lacking a “G” at the opposite position of the deleted “C”), but still gave proper exon 105 skipping in these wild type fibroblasts in which AON8 is not 100% complementary to the exon 105 mRNA.

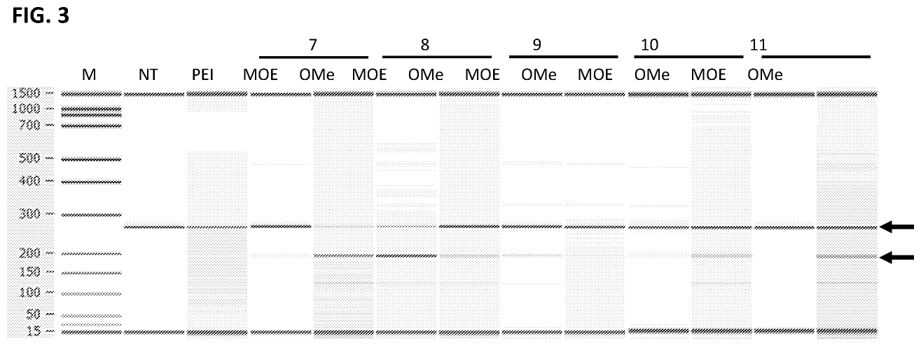

In a follow-up experiment, the 2′-OMe modification was compared to 2′-MOE for each of AON7, -8, -9, -10, and -11 (see Table 1) in a similar PCR experiment (using the same procedures as described above). The AONs were either fully modified at each positioned with 2′-OMe or fully modified at each position with 2′-MOE. The results are shown in FIG. 3. Here, some skipping could be detected with AON7 (2′-MOE). The results also reveal that where AON7 with 2′-OMe outperformed its 2′-MOE equivalent, for AON8 this was the opposite.

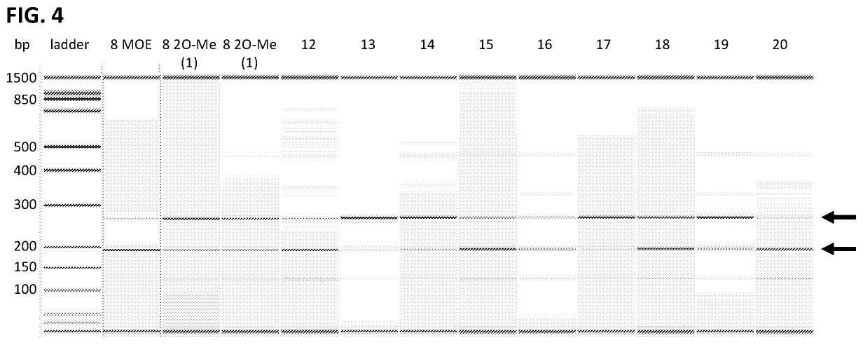

In a next experiment, a new set of AONs was generated, all based on AON8 and using also AONs that were 100% complementary with the wild type exon 105 mRNA at the c.7864delC position (AONs 12, 15, 16, see FIG. 1 and Table 1). For this, wild type fibroblasts were transfected with AON12, -13, -14, -15, -16, -17, -18, -19, and -20 (all carrying the 2′-MOE modification in full), and compared to the 2′-MOE- and 2′-OMe-modified versions of AON8. The results are shown in FIG. 4. The best performing AONs were AON12, AON15, AON16, AON18, and AON20. As can be seen in FIG. 1, AONs 12, 15, and 16 are 100% complementary to the wild type exon 105 mRNA. The effect of AONs 8, 9, 13, 14, 17, and 19 on c.7864delC mutated mRNA is determined in another experiment. AON18 is complementary to a region away from the c.7864delC mutation. It is concluded that AONs 8, 12, 15, 16, 18, and 20 (with 2′-MOE modifications) all outperform the AONs from the prior art.

### Example 2: Quantification of Exon 105 Exclusion from Human COL7A1 Pre-mRNA Using ddPCR

To better quantify the amount of C74105 mRNA after transfections with different AONs as disclosed herein, the inventors of the present invention developed a droplet digital PCR (ddPCR) assay.

Exon 105 skipping was measured using two duplex assays. In assay 1 the total amount of COL7A1 in the samples was measured (COL7A1 reference assay with a FAM label) and in assay 2 a primer and probe set specific for the skipped exon 105 (COL7A1 exon 105 skipping assay with a FAM label) was used. In order to normalize the samples both assays were duplexed using GusB as reference gene with a HEX label. The following primers and probe sequences were used:

GusB
Forward primer:
(SEQ ID NO: 65)
5′-GTTTTTGATCCAGACCCAGATG-3′
Reverse primer:
(SEQ ID NO: 66)
5′-GCCCATTATTCAGAGCGAGTA-3′
Probe (HEX):
(SEQ ID NO: 67)
5′-TGCAGGGTTTCACCAGGATCCAC-3′
C7
Forward primer:
(SEQ ID NO: 68)
5′-TCGGTTGCTGGAAACTGC-3′
Reverse primer:
(SEQ ID NO: 69)
5′- CACAGGCAGGAAGCTACC-3′
Probe (FAM):
(SEQ ID NO: 70)
5′-ATCAAGGCATCTGCCCTGCGGGAG-3′
C7-Δ105
Forward primer:
(SEQ ID NO: 63)
5′-GTGACAAAGGACCTCGGGG-3′
Reverse primer:
(SEQ ID NO: 64)
5′-CTCCATCAAGGCCACAGGC-3′
Probe (FAM):
(SEQ ID NO: 71)
5′-ACTCCCCGTTCACCCGGGTCAC-3′

Duplex ddPCR was performed using the ddPCR super mix for probes (no dUTP) (Biorad) with 4 μL cDNA template and primers and probes (end concentration of 0.2 μM) in a total volume of 20 μL. Droplets were generated using the QX200 droplet generator (Biorad) and PCR was performed for 40 cycles using an annealing temperature of 62° C. using the T100 thermal cycler (Biorad). After PCR the droplets were analyzed in the QX200 droplet reader (Biorad), counting the fluorescent signals from the single labelled, double labelled, and negative droplets. Exon 105 skipping % was calculated using the following formula:

$$Exon 105 skip ( \% ) = \frac{( \frac{Copies exon 105 skip}{Copies GusB} )}{( \frac{Copies C 7 ref}{Copies GusB} )}$$

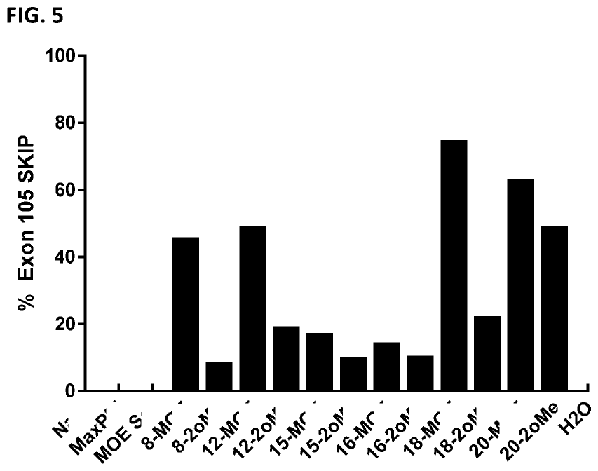

The experiments were first performed using wild type fibroblasts. AONs 8, 12, 15, 16, 18 and 20 were tested first, in both their 2′-OMe and 2′-MOE versions. FIG. 5 shows the results of this initial ddPCR screen, and clearly reveals the superiority of the 2′-MOE modification over the 2′-OMe modification in this setup, with in each case a higher percentage of skipping when the 2′-MOE version was used in comparison to its 2′-OMe counterpart. Although AON8 was still a good performer, AON18 and AON20 gave even higher percentages of skip, up to 80%. The clear difference between AON8 with 2′-MOE and AON8 with 2′-OMe reflects the results shown in FIGS. 3 and 4, in which also the 2′-MOE version appeared to give better results.

### Example 3: Quantification of Exon 105 Skipping Using Patient-Derived Fibroblasts

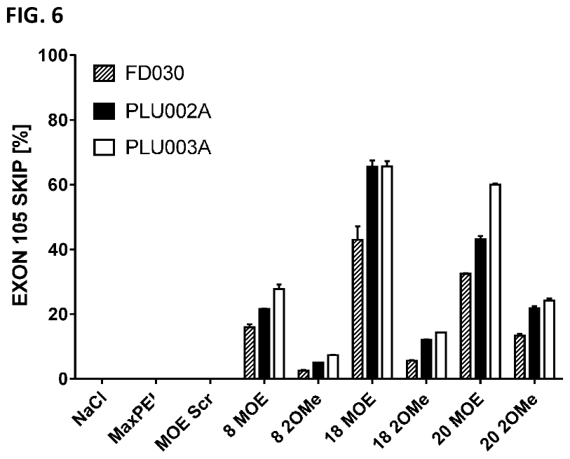

The ddPCR assay was also used to determine the percentage of exon 105 skipping in wild type fibroblasts (FD030) and in fibroblasts obtained from a DEB patient carrying the c.7864delC mutation (PLU002A) and from a human subject, not suffering from DEB but identified as a carrier of the c.7864delC mutation (PLU003A). In this experiment AON8, AON18 and AON20, both in the 2′-OMe and the 2′-MOE versions were tested, while non-transfection and a scrambled 2′-MOE AON were included as negative controls. Experimental setup was as described in example 2. The results are shown in FIG. 6. These reveal that in all three cell types AON18 and AON20, both fully modified with 2′-MOE outperformed their 2′-OMe counterparts and AON8. No drastic differences were observed in results obtained in patient material versus carrier material, but the percentage of skipping in patient and carrier fibroblasts was for each of the AONs higher than in wild type fibroblasts.

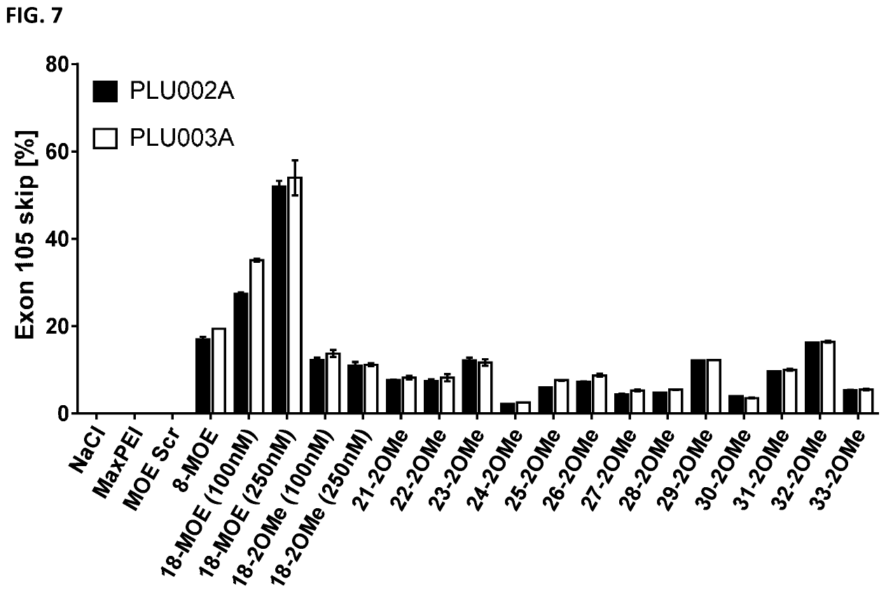

Next, it was decided to generate yet another set of AONs (AONs 21-33; see Table 1 and FIG. 1), all targeting the complementarity region of AONs 8-20. The sequence of AON21 was already disclosed in WO 2017/078526, although that particular oligonucleotide was not shown to be tested for exon skipping efficiency therein. AONs 21-33 (all with full 2′-OMe modifications) were also tested in PLU002A and PLU003A fibroblasts. AON18 with the 2′-MOE and 2′-OMe modifications were tested both in two different concentrations and compared to the newly generated AONs and to AON8. The results are shown in FIG. 7 and reveal that none of the newly generated AONs (all carrying the 2′-OMe modification) was able to reach the percentage of skipping obtained with AON18 carrying the 2′-MOE modification, although AON32 reached a level that was comparable to AON8.

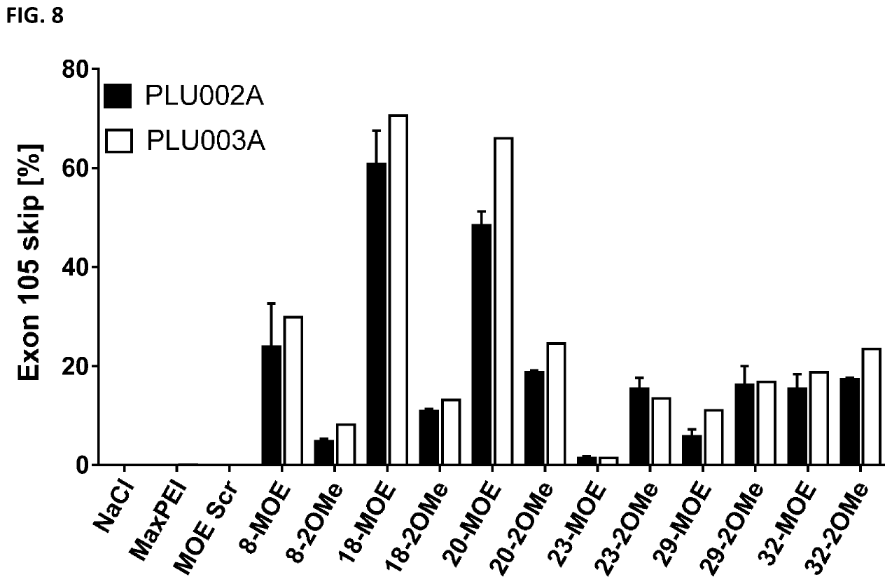

For AON23, AON29, and AON32, all terminating at their 3′ end with . . . CAUCUCC-3′, indicating that having this particular terminus in a human COL7A1 exon 105 skipping oligonucleotide provides superior efficiency, also a 2′-MOE version was generated and compared to the 2′-MOE versions of AON8, AON18 and AON20, in a similar ddPCR experiment, using PLU002A and PLU003A fibroblasts. Also AON20 comprises the . . . CAUCUCC-3′ terminus. FIG. 8 shows the results of this experiment and reveals that especially AON8, AON18, AON20, AON29 and AON32 give good exon 105 skipping results, with AON8, AON32, AON20 and AON18 performing best.

### Example 4: Quantification of Exon 105 Skipping Upon Transfecting of New and Known AONs in DEB Fibroblasts

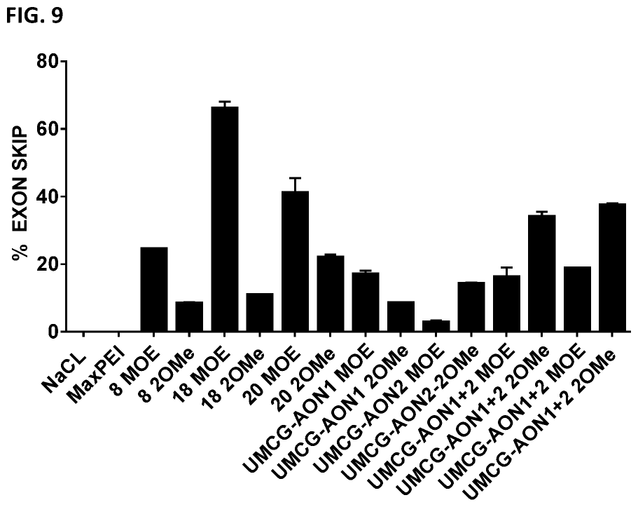

To assess the efficiency of exon 105 skipping by the AONs of the present invention in comparison to the two known AONs of the prior art, a similar transfection in PLU002A fibroblasts was performed with AON8, AON18, AON20, all fully modified with either 2′-OMe, or 2′-MOE, and compared to separate transfections with UMCG-AON1 and UMCG-AON2, both fully modified with either 2′-OMe, or 2′-MOE, as well as to combined transfections wherein UMCG-AON1 (2′-OMe) was co-transfected with UMCG-AON2 (2′OMe), or wherein UMCG-AON1 (2′-MOE) was co-transfected with UMCG-AON2 (2′-MOE). Final concentration of the total AON that was transfected was in all cases 250 nM also in the combined co-transfections). Cell cultures, transfections, mRNA isolation, and ddPCR procedures were as described as above. Results are given in FIG. 9. Clearly, the 2′-MOE versions of AON8, AON18 and AON20 outperformed their 2′-OMe counterparts. For all these three AONs, their 2′-MOE version also outperformed the 2′-MOE versions as well as the 2′-OMe versions of UMCG-AON1 and UMCG-AON2, when transfected as a sole compound. Importantly, the sole transfection of AON18 (2′-MOE) and AON20 (2′-MOE) even gave more efficient exon 105 skipping than the combined co-transfection of UMCG-AON1/UMCG-AON2 in both 2′-OMe and 2′-MOE versions (each performed in duplo, as indicated beneath the bars). This clearly shows the beneficial properties of the new AONs disclosed herein, that can be used as a single active compound to reach appropriate exon 105 skipping from the human COL7A1 pre-mRNA in cells obtained from a human patient suffering from DEB.

### Example 5: Design and Characterization of Additional AONs for Exon 105 Exclusion from Human COL7A1 Pre-mRNA

To further identify AONs useful for mediating exon 105 skipping in human COL7A1 pre-mRNA, 21 additional AONs were designed and assessed for their activity. All AONs were modified with phosphorothioate linkages connecting the nucleosides, and further modified as in one or more of the following: MOE (fully 2′-MOE-modified at the sugar moiety); OMe (fully 2′-OMe-modified at the sugar moiety); MOE/1×LNA (5′- and 3′-terminal nucleotides LNA-modified, all other nucleotides 2′-MOE-modified at the sugar moiety); OMe/1×LNA (5′- and 3′-terminal nucleotides LNA-modified, all other nucleotides 2′-OMe-modified at the sugar moiety); MOE/2×LNA (two 5′- and two 3′-terminal nucleotides LNA-modified, all other nucleotides 2′-MOE-modified at the sugar moiety); OMe/2×LNA (two 5′- and two 3′-terminal nucleotides LNA-modified, all other nucleotides 2′-OMe-modified at the sugar moiety). Table 2 shows the additional AONs that were assessed, with their SEQ ID NOs and in what modified versions they were tested. FIG. 1 shows the respective positions of all these AONs in relation to their complementary sequence in exon 105 (top).

TABLE 2
Additional AONs tested for skipping of exon 105. AONs were tested with
different chemical modifications as indicated by an “x” in the respective column.

| | | Modifications tested | | | | | |
|---|---|---|---|---|---|---|---|
| | SEQ ID | MOE + | OMe + | MOE/ | OMe/ | MOE/ | OMe/ |
| AON | NO | PS¹ | PS² | 1 × LNA + PS³ | 1 × LNA + PS⁴ | 2 × LNA + PS⁵ | 2 × LNA + PS⁶ |
| AON34 | 39 | x | | | | | |
| AON47 | 52 | | x | | | | |
| UMCG- | 3 | x | x | | | | |
| AON1 | | | | | | | |
| AON48 | 53 | | x | | | | |
| AON54 | 59 | x | x | x | x | | |
| AON46 | 51 | | x | | | | |
| AON55 | 60 | x | x | x | x | | |
| AON53 | 58 | x | x | x | x | | |
| AON39 | 44 | x | x | x | x | x | x |
| AON45 | 50 | | x | | | | |
| AON44 | 49 | | x | | | | |
| AON52 | 57 | x | x | | | | |
| UMCG- | 4 | x | x | | | | |
| AON2 | | | | | | | |
| AON51 | 56 | x | x | | | | |
| AON43 | 48 | x | x | x | x | | |
| AON37 | 42 | x | x | x | x | x | x |
| AON38 | 43 | x | x | | | | |
| AON40 | 45 | x | x | | | | |
| AON36 | 41 | x | | | | | |
| AON42 | 47 | x | x | | | | |
| AON41 | 46 | x | x | | | | |
| AON50 | 55 | | x | | | | |
| AON49 | 54 | | x | | | | |

¹MOE + PS: Fully 2′-MOE-modified with PS backbone.
²OMe + PS: Fully 2′-OMe-modified with PS backbone.
³MOE/1 × LNA + PS: 5′- and 3′-terminal nucleotides LNA-modified, all other nucleotides 2′-MOE-modified, with full PS backbone.
⁴OMe/1 × LNA + PS: 5′- and 3′-terminal nucleotides LNA-modified, all other nucleotides 2′-OMe-modified, with full PS backbone.
⁵MOE/2 × LNA + PS: two 5′- and two 3′-terminal nucleotides LNA-modified, all other nucleotides 2′-MOE-modified, with full PS backbone.
⁶OMe/2 × LNA + PS: two 5′- and two 3′-terminal nucleotides LNA-modified, all other nucleotides 2′-OMe-modified, with full PS backbone.

### Transfection-Mediated Delivery of AONs in Human Fibroblasts

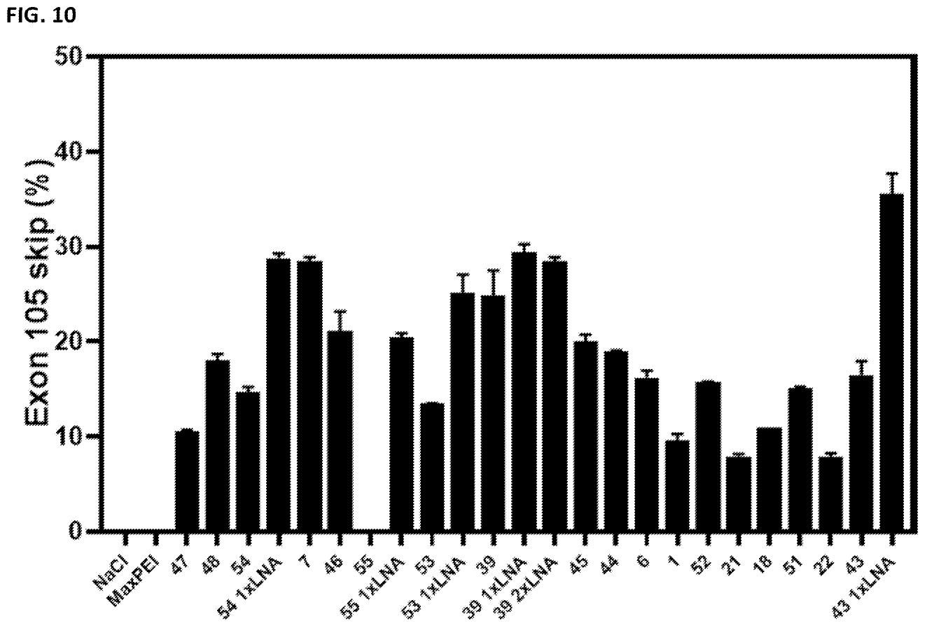

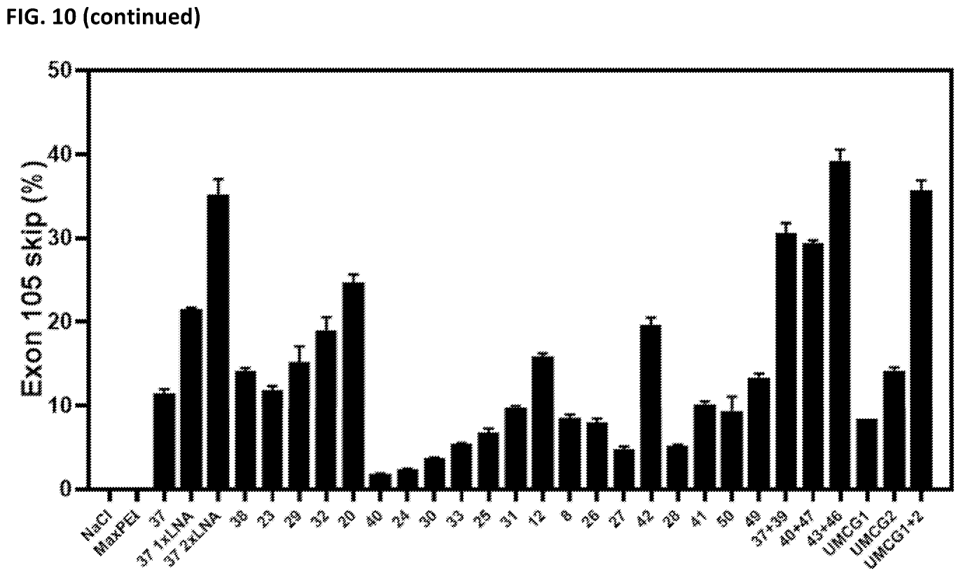

In a first experiment, the frequency of exon 105 skipping in human COL7A1 pre-mRNA from human fibroblasts transfected with T-OMe variants of the indicated AONs was assayed by ddPCR as described above (FIG. 10). Variants of AON37, AON39, AON43, AON53, AON54, and AON55 tested included those having their 5′- and 3′-terminal nucleotides LNA-modified (1×LNA) with all other nucleotides T-OMe-modified. Variants of AON37 and AON39 tested included those having their two 5′- and two 3′-terminal nucleotides LNA-modified (2×LNA) with all other nucleotides 2′-OMe-modified. NaCl and transfection reagent only (MaxPEI) conditions were included as negative controls, and 2′-OMe-modified UMCG-AON1 (UMCG1) and UMCG-AON2 (UMCG2) were included for reference. The following pairs of AONs were also tested in combination: AON37+AON39; AON40+AON47; AON43+AON46; and UMCG1+UMCG2. As shown in FIG. 10, many of the experimental T-OMe-modified AONs had an improved frequency of exon 105 skipping as compared to UMCG1 or UMCG2. Introduction of the 1×LNA modification resulted in improved exon 105 skipping frequency for all AONs tested, and for at least one AON the exon 105 skipping frequency increased further with the 2×LNA modification (see AON37, AON37 1×LNA, and AON37 2×LNA in FIG. 10). Notably, whereas AON55 (having only the T-OMe modification) showed no activity for exon 105 skipping, further introduction of the 1×LNA modification resulted in an AON capable of mediating about 20% skipping of exon 105, greater than that observed for either UMCG1 or UMCG2 in this experiment. The two single AONs with the highest activity were AON43 1×LNA and AON37 2×LNA, both showing about 35% skipping of exon 105. The highest activity was observed for the combination of AON43+AON46 at about 40% skipping of exon 105.

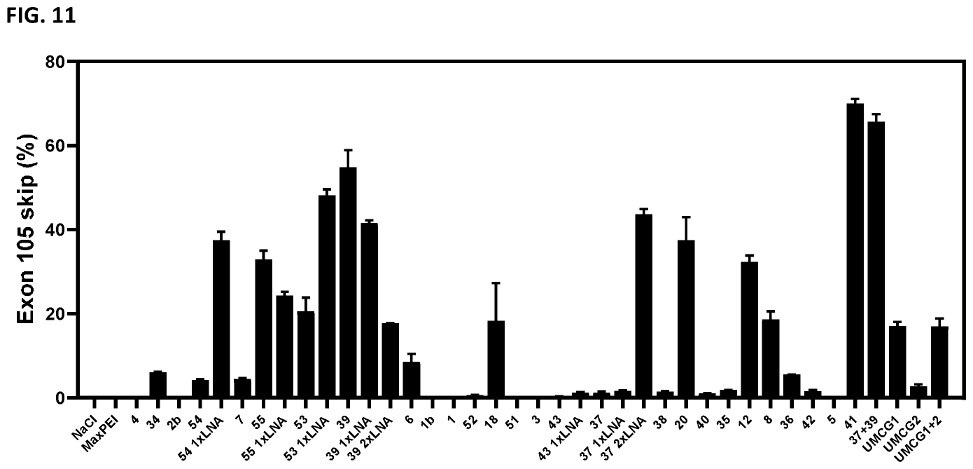

In another experiment, the frequency of exon 105 skipping in human COL7A1 pre-mRNA from human fibroblasts transfected with 2′-MOE variants of the indicated AONs was assayed by ddPCR as described above (FIG. 11). Variants of AON37, AON39, AON43, AON53, AON54, and AON55 tested included those having their 5′- and 3′-terminal nucleotides LNA-modified (1×LNA) with all other nucleotides 2′-MOE-modified. Variants of AON37 and AON39 tested included those having their two 5′- and two 3′-terminal nucleotides LNA-modified (2×LNA) with all other nucleotides 2′-MOE-modified. NaCl and transfection reagent only (MaxPEI) conditions were included as negative controls, and T-MOE-modified UMCG-AON1 (UMCG1) and UMCG-AON2 (UMCG2) were included for reference. The following pairs of AONs were also tested in combination: AON37+AON39; and UMCG1+UMCG2. As shown in FIG. 11, many of the experimental 2′-MOE-modified AONs had an improved frequency of exon 105 skipping as compared to UMCG1 or UMCG2. Introduction of the 1×LNA modification resulted in improved exon 105 skipping frequency for some of the AONs tested (see AON54 1×LNA and AON53 1×LNA in FIG. 11), though others showed reduced activity (see AON55 1×LNA and AON39 1×LNA in FIG. 11) or little to no change (see AON37 1×LNA and AON43 1×LNA in FIG. 11). In one case, where there was little to no activity with either the 2′-MOE modification alone or in combination with the 1×LNA modification, the exon 105 skipping frequency increased to over 40% with the 2×LNA modification (see AON37, AON37 1×LNA, and AON37 2×LNA in FIG. 11), greater than that observed for either UMCG1 or UMCG2 in this experiment. In another case, where the activity decreased with introduction of the 1×LNA modification, the exon 105 skipping frequency decreased further with the 2×LNA modification (see AON39 2×LNA in FIG. 11). The highest activity was observed for AON41 alone, which showed about 70% skipping of exon 105. The combination of AON37+AON39 showed similar activity at slightly less than about 70% skipping of exon 105.

### Gymnotic Delivery of AONs in HeLa Cells

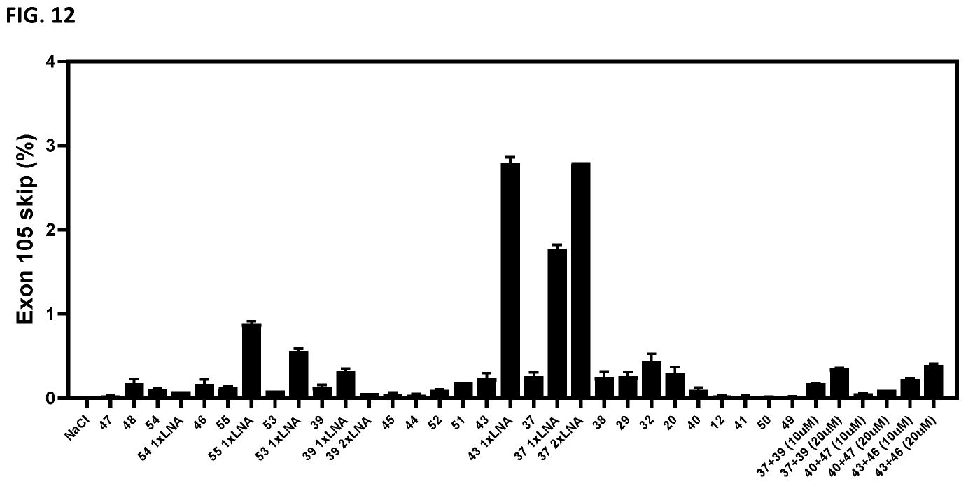

In order to further characterize the AONs, their ability to mediate skipping of exon 105 in cells following gymnotic delivery was assessed. The frequency of exon 105 skipping in human COL7A1 pre-mRNA from HeLa cells following gymnotic uptake of 2′-OMe variants of the indicated AONs was assayed by ddPCR as described above (FIG. 12). Briefly, 5.0×10⁴HeLa cells were seeded per well in 12-well plates and subsequently cultured in DMEM supplemented with 10% FBS, 100 U/mL penicillin and 0.1 mg/mL streptomycin at 37° C. and 5.0% CO₂. The next day, cells were treated by adding AON directly to the wells to a final concentration of 3-50 μM. The cells were harvested after 72 hours of treatment and stored at −80° C. prior to RNA analysis as described above. Variants of AON37, AON39, AON43, AON53, AON54, and AON55 tested included those having their 5′- and 3′-terminal nucleotides LNA-modified (1×LNA) with all other nucleotides 2′-OMe-modified. Variants of AON37 and AON39 tested included those having their two 5′- and two 3′-terminal nucleotides LNA-modified (2×LNA) with all other nucleotides 2′-OMe-modified. An NaCl-only condition was included as a negative control. The following pairs of AONs were also tested in combination: AON37+AON39; AON40+AON47; and AON43+AON46. As shown in FIG. 12, many of the experimental AONs with only the 2′-OMe modification had a relatively low frequency of exon 105 skipping as compared to the respective AON further including either the 1×LNA or 2×LNA modification. Introduction of the 1×LNA modification resulted in improved exon 105 skipping frequency for AON55, AON53, AON39, AON43, and AON37. Introduction of the 2×LNA modification resulted in further improved exon 105 skipping frequency for AON37. Whereas none of the T-OMe-only modified AONs were capable of mediating more than about 0.5% skipping of exon 105, further modification with 1×LNA or 2×LNA allowed for up to about 3% skipping of exon 105 in some cases (see AON43 1×LNA and 37 2×LNA in FIG. 12).

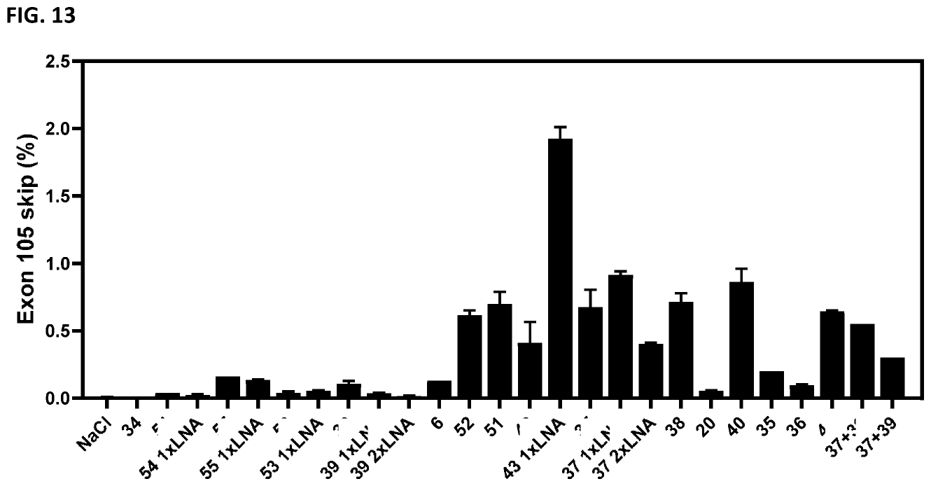

In another experiment, the frequency of exon 105 skipping in human COL7A1 pre-mRNA from HeLa cells following gymnotic uptake of T-MOE variants of the indicated AONs was assayed by ddPCR as described above (FIG. 13). Variants of AON37, AON39, AON43, AON53, AON54, and AON55 tested included those having their 5′- and 3′-terminal nucleotides LNA-modified (1×LNA) with all other nucleotides 2′-MOE-modified. Variants of AON37 and AON39 tested included those having their two 5′- and two 3′-terminal nucleotides LNA-modified (2×LNA) with all other nucleotides 2′-MOE-modified. An NaCl-only condition was included as a negative control. The following pairs of AONs were also tested in combination: AON37+AON39. As shown in FIG. 13, there did not appear to be much benefit with regard to exon 105 skipping activity for the LNA modifications of the T-MOE AON variants, except for AON43, where the frequency of exon 105 skipping increased from less than about 0.5% to about 2% with the introduction of the 1×LNA modification. None of the experimental AONs with only the T-MOE modification were able to facilitate at least about 1% exon 105 skipping. Only one AON, AON43 1×LNA, demonstrated such activity, showing about 2% skipping of exon 105.

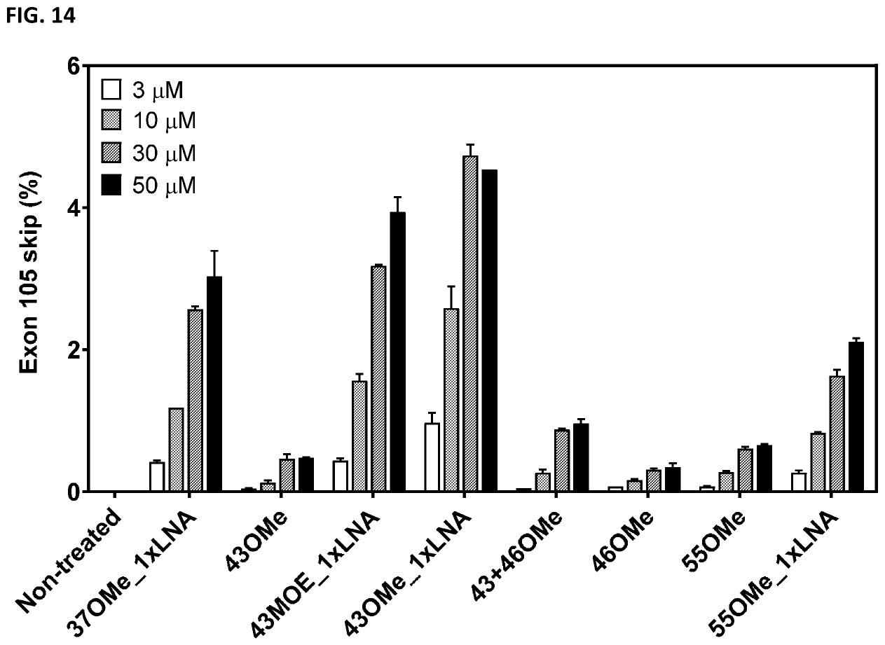

A subset of the AONs, including some of the most active variants as determined in the previous studies (AON37 OMe/1×LNA, AON43 OMe/1×LNA, and AON43 MOE/1×LNA), were further characterized by evaluating their dose-response at various concentrations (3 μM, 10 μM, 30 μM, and 50 μM) on the frequency of exon 105 skipping in human COL7A1 pre-mRNA from HeLa cells following gymnotic uptake of the AONs, as assayed by ddPCR (FIG. 14). As shown in FIG. 14, all of the AONs tested showed a positive dose-response curve, providing strong evidence for a causal relationship between treatment with the AONs and skipping of exon 105.

### Human Skin Equivalent (HSE) Models of Wounding

To evaluate the activity of the AONs in a three-dimensional model of skin wounding, human skin equivalent (HSE) models were prepared and treated with AONs following superficial wounding.

To prepare HSEs, first dermal equivalents were generated by seeding rat-tail collagen at about 4 mg/mL in 20 mM Acetic Acid with 8.0×10⁴ primary fibroblasts (passage number 4-6) in 6-well filter inserts and incubated for one week under submerged conditions in standard fibroblast medium DMEM supplemented with 5% FBS, 100 U/mL penicillin, and 0.1 mg/mL streptomycin. Subsequently, 1.5×10⁵ or 3.0×10⁵ (passage number 1) keratinocytes were seeded dropwise in 100 μL seeding medium (DMEM:Ham's F12 medium (3:1) supplemented with 5% FBS, 1 μM hydrocortisone, 1 μM isoproterenol, 0.1 μM insulin, 100 U/mL penicillin, and 0.1 mg/mL streptomycin) onto the dermal equivalents. Two hours after seeding, an additional 1.5 mL of seeding medium was added on top of each of the models. HSEs were incubated under submerged conditions at 37° C. and 7.3% CO₂ in seeding medium. After 2 days, FBS was reduced to 1%. Two days thereafter, FBS was omitted and the HSEs were cultured at the air-liquid interface for 12 days before wounding and treatment. During this period, HSEs were cultured in differentiation medium (DMEM:Ham's F12 medium (3:1) supplemented with 100 U/mL penicillin, 0.1 mg/mL streptomycin, 1 μM hydrocortisone, 1 μM isoproterenol, 0.1 μM insulin, 10 mM 1-serine, 10 μM 1-carnitine, and 53 nM selenious acid). Medium was refreshed twice a week; at that time the following supplements were freshly added: 100 μg/ml ascorbic acid, 1 μM DL-α-tocopherolacetate in (3-dextrine (final concentration), 24 μM bovine serum albumin, and a free fatty acid supplement that contained 25 μM palmitic, 30 μM linoleic acid, and 7 μM arachidonic acid.

Superficial wounds in the HSEs were made by removing the epidermis. Briefly, a 5-6 mm wide sterile template strip of filter paper was placed on the center surface of each HSE. Linear incisions were made on both sides of the template strip and the epidermis beneath each template strip was removed using sterile forceps. This resulted in a superficial wound on an intact dermal equivalent harboring fibroblasts.

The HSEs were then treated with AON formulated in a 0.75% carbomer hydrogel at a concentration of 10 mg AON/g gel. The HSEs were treated three times a week (Mondays, Wednesdays, and Fridays) for three weeks for a total of nine treatments. Application of the amount of hydrogel with or without AON was performed as standardized as possible. To this end, for each HSE, 50 mg/cm² (depending on the wound size; 50 μL (=50 mg) for a 5 mm wound, and 75 μL (=75 mg) for a 6 mm wound, D035 and D090) of the hydrogel was weighed on a piece of parafilm and applied onto the wounded HSE with a spatula or measured and applied onto the wounded HSE using a positive displacement pipette. The hydrogel was spread with a spatula over the entire surface of the wound bed, leaving the wound edges free of hydrogel. After treatment, all HSEs were placed back into culture (37° C., 7.3% CO₂) until harvesting.

The HSE models were harvested three weeks after wounding and initiation of treatment. Four different samples were collected in RNAlater for RNA isolation; a sample containing both epidermis and dermis (RNA D+E), a sample containing only the epidermis (RNA E), a dermal sample from the wound bed (RNA DW), and a dermal sample from underneath the intact epidermis (RNA D). The samples were stored overnight at 4° C. and transferred to −80° C. thereafter. RNA was isolated using the Rneasy plus universal mini kit (Qiagen) and the RNA was stored at −80° C. cDNA synthesis was performed using 300 ng RNA. The cDNA synthesis was performed for 30 minutes at 42° C. using random hexamers (provided by the Verso kit, Thermo Fisher) to synthesize the first strand cDNA. The cDNA was stored at −20° C. Exon 105 skipping was measured using the same method as described above.

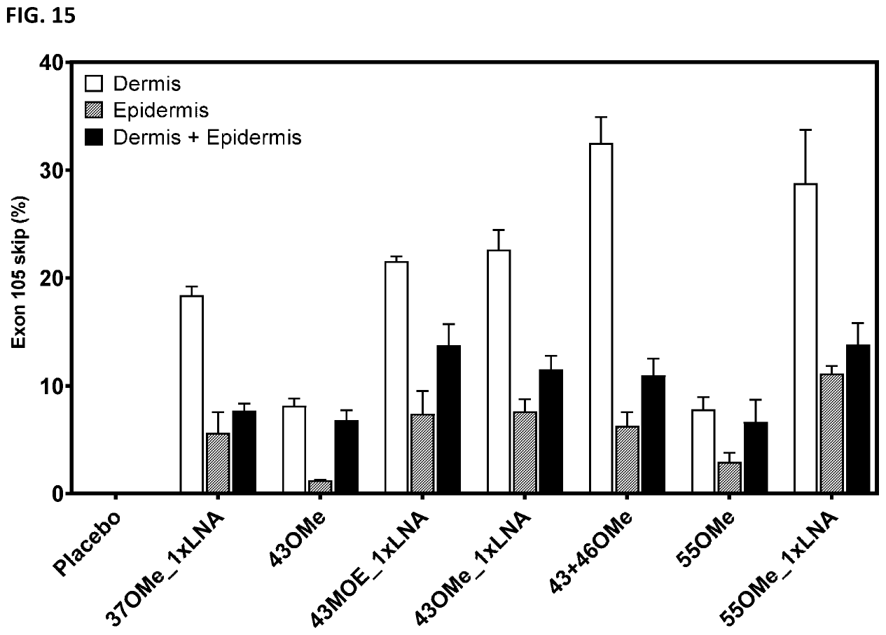

The frequency of exon 105 skipping in human COL7A1 pre-mRNA from dermis, epidermis, or dermis+epidermis of HSE models of wounding following treatment for three weeks with a subset of AONs as indicated, including some of the most active variants as determined in the previous studies (A0N37 OMe/1×LNA, AON43 OMe/1×LNA, and AON43 MOE/1×LNA), in a carbomer hydrogel formulation was assayed by ddPCR as described above (FIG. 15). As shown in FIG. 15, skipping of exon 105 was observed in both dermis and epidermis of the HSE models of wounding treated with the AONs, with a frequency of about 20% or greater in dermis for most of the AONs tested. These results support that topical administration to skin wounds of human subject suffering from dystrophic epidermolysis bullosa (DEB) is a feasible approach to deliver the AONs described herein to target cells in the skin. Moreover, these findings support that a formulation resembling EB standard of care seems suitable for delivery of the AONs.

Sequence Listing

| SEQ | | |
|---|---|---|
| ID | | |
| NO | Sequence | Description |
| 1 | GGAUCCCCAGGAAAGGAUGGAGU | Wild-type pre-mRNA |
| | GCCUGGUAUCCGAGGAGAAAAAG | sequence of |
| | GAGAUGUUGGCUUCAUGGGUCCC | human COL7A1 |
| | CGGGGCCUCAAG | exon 105 |
| 2 | ucucccugcuagGGAUCCCCAGG | Human COL7A1 pre- |
| | AAAGGAUGGAGUGCCUGGUAUCC | mRNA fragment |
| | GAGGAGAAAAAGGAGAUGUUGGC | including |
| | UUCAUGGGUCCCCGGGGCCUCAA | wild-type |
| | Ggua | exon 105 |
| 3 | GAUACCAGGCACUCCAUCCU | AON UMCG-A0N1 |
| 4 | CAUGAAGCCAACAUCUCCUU | AON UMCG-A0N2 |
| 5 | ACAUCUCCUUUUUCUCCUCG | AON 1 |
| 6 | UCCUUUUUCUCCUCGGAUAC | AON lb |
| 7 | GGCACUCCAUCCUUUCCUGG | AON 2 |
| 8 | AGGCACUCCAUCCUUUCCUG | AON 2b |
| 9 | CCAUGAAGCCAACAUCUCCU | AON 3 |
| 10 | GGAUCCCUAGCAGGGAGA | AON 4 |
| 11 | UUCUUUCCUACCUUGAGGCC | AON 5 |
| 12 | CUUUUUCUCCUCGGAUACCA | AON 6 |
| 13 | CUCCUCGGAUACCAGGCACU | AON 7 |
| | CCAU | |
| 14 | CCGGGACCCAUGAAGCCAAC | AON 8 |
| | AUCU | |
| 15 | CCGGGACCCAUGAAGCCAAC | AON 9 |
| 16 | GACCCAUGAAGCCAACAUCU | AON 10/35 |
| 17 | GGGACCCAUGAAGCCAACAU | AON 11 |
| 18 | CGGGGACCCAUGAAGCCAAC | AON 12 |
| | AUCU | |
| 19 | CCCGGGACCCAUGAAGCCAA | AON 13 |
| | CAUC | |
| 20 | CCGGGACCCAUGAAGCCAAC | AON 14 |
| | AUC | |
| 21 | CGGGGACCCAUGAAGCCAAC | AON 15 |
| 22 | CCGGGGACCCAUGAAGCCAA | AON 16 |
| 23 | CCGGGACCCAUGAAGCCAAC | AON 17 |
| | AU | |
| 24 | GACCCAUGAAGCCAACAU | AON 18 |
| | CUCCUU | |
| 25 | GGCCCCGGGACCCAUGAA | AON 19 |
| | GCCAAC | |
| 26 | GGGACCCAUGAAGCCAACAUC | AON 20 |
| | UCC | |
| 27 | CCAUGAAGCCAACAUCUCCUU | AON 21 |
| 28 | CCCAUGAAGCCAACAUCUCCU | AON 22 |
| 29 | ACCCAUGAAGCCAACAUCUCC | AON 23 |
| 30 | GACCCAUGAAGCCAACAUCUC | AON 24 |
| 31 | GGACCCAUGAAGCCAACAUCU | AON 25 |
| 32 | GGGACCCAUGAAGCCAACAUC | AON 26 |
| 33 | CGGGACCCAUGAAGCCAACAU | AON 27 |
| 34 | CCGGGACCCAUGAAGCCAACA | AON 28 |
| 35 | GACCCAUGAAGCCAACAUCUCC | AON 29 |
| 36 | GGACCCAUGAAGCCAACAUCUC | AON 30 |
| 37 | GGGACCCAUGAAGCCAACAUCU | AON 31 |
| 38 | GGACCCAUGAAGCCAACAUCUCC | AON 32 |
| 39 | GGGACCCAUGAAGCCAACAUCUC | AON 33 |
| 40 | CAUCCUUUCCUGGGGAUCC | AON 34 |
| 41 | GGACCCAUGAAGCCAACAUC | AON 36 |
| 42 | CCAUGAAGCCAACAUCUCC | AON 37 |
| 43 | CCCAUGAAGCCAACAUCUCC | AON 38 |
| 44 | CUCCUCGGAUACCAGGCACUC | AON 39 |
| 45 | CCAUGAAGCCAACAUCUC | AON 40 |
| 46 | GUUUCUUUCCUACCUUGAGG | AON 41 |
| 47 | GGACCCAUGAAGCCAACA | AON 42 |
| 48 | CAUGAAGCCAACAUCUCC | AON 43 |
| 49 | UUCUCCUCGGAUACCAGGCA | AON 44 |
| 50 | UCUCCUCGGAUACCAGGCAC | AON 45 |
| 51 | CCUCGGAUACCAGGCACUCC | AON 46 |
| 52 | CCAGGCACUCCAUCCUUUCC | AON 47 |
| 53 | GGAUACCAGGCACUCCAUCC | AON 48 |
| 54 | CUUGUUUCUUUCCUACCUUG | AON 49 |
| 55 | UGUUUCUUUCCUACCUUGAG | AON 50 |
| 56 | CAUGAAGCCAACAUCUCCU | AON 51 |
| 57 | AUGAAGCCAACAUCUCCUU | AON 52 |
| 58 | CCUCGGAUACCAGGCACUC | AON 53 |
| 59 | CGGAUACCAGGCACUCCAUC | AON 54 |
| 60 | CUCGGAUACCAGGCACUC | AON 55 |
| 61 | TTGGCTTCATGGG | COL7A1 |
| | | pre-mRNA |
| | | exon 105 |
| | | region 1 |
| | | (E105-R1) |
| 62 | CCCAUGAAGCCAA | AON sequence |
| | | targeting |
| | | E105-R1 |
| 63 | GTGACAAAGGACCTCGGGG | cDNA Forward |
| | | primer; |
| | | C7-Δ105 |
| | | Forward |
| | | primer |
| 64 | CTCCATCAAGGCCACAGGC | cDNA Reverse |
| | | primer; |
| | | C7-Δ105 Reverse |
| | | primer |
| 65 | GTTTTTGATCCAGACCCA | GusB Forward |
| | GATG | primer |
| 66 | GCCCATTATTCAGAGCGA | GusB Reverse |
| | GTA | primer |
| 67 | TGCAGGGTTTCACCAGG | GusB Probe |
| | ATCCAC | (HEX) |
| 68 | TCGGTTGCTGGAAACTGC | C7 Forward |
| | | primer |
| 69 | CACAGGCAGGAAGCTACC | C7 Reverse |
| | | primer |
| 70 | ATCAAGGCATCTGCCC | C7 Probe (FAM) |
| | TGCGGGAG | |
| 71 | ACTCCCCGTTCACCCGGGTCAC | C7-Δ105 Probe |
| | | (FAM) |

1. An antisense oligonucleotide (AON) capable of preventing or reducing exon 105 inclusion into a human collagen type VII alpha 1 chain (COL7A1) mRNA when the mRNA is produced by splicing from a pre-mRNA in a cell, wherein the AON comprises or consists of a nucleotide sequence that is selected from the group consisting of SEQ ID NOs: 48, 42, 60, 44, 46, 51, 52, 58, 59, 5-41, 43, 45, 47, 49, 50, and 53-57.

2. The AON according to claim 1, wherein the AON comprises or consists of a nucleotide sequence that is selected from the group consisting of SEQ ID NOs: 48, 42, 60, 44, 46, 51, 52, 58, and 59.

3. The AON according to claim 1, wherein the AON comprises or consists of a nucleotide sequence that is selected from the group consisting of SEQ ID NOs: 24, 26, 14, 15, 16, 17, 18, 19, 20, 21, 22, 23, 25, 28, 29, 30, 31, 32, 33, 34, 35, 36, 37, 38, 39, 5, 6, 8, 9, 10, 11, 12, and 13.

4. An AON capable of preventing or reducing exon 105 inclusion into a human COL7A1 mRNA when the mRNA is produced by splicing from a pre-mRNA in a cell, wherein the AON comprises or consists of a nucleotide sequence that is complementary to a target nucleotide sequence in a COL7A1 pre-mRNA corresponding to any one of SEQ ID NOs: 48, 42, 60, 44, 46, 51, 52, 58, 59, 5-41, 43, 45, 47, 49, 50, and 53-57.

5. The AON according to claim 4, wherein the AON comprises or consists of a nucleotide sequence that is complementary to a target nucleotide sequence in a COL7A1 pre-mRNA corresponding to any one of SEQ ID NOs: 48, 42, 60, 44, 46, 51, 52, 58, and 59.

6. The AON according to claim 4, wherein the AON comprises or consists of a nucleotide sequence that is complementary to a target nucleotide sequence in a COL7A1 pre-mRNA corresponding to SEQ ID NOs: 24, 26, 14, 15, 16, 17, 18, 19, 20, 21, 22, 23, 25, 28, 29, 30, 31, 32, 33, 34, 35, 36, 37, 38, 39, 5, 6, 8, 9, 10, 11, 12, and 13.

7. The AON according to any one of claims 1 to 6, wherein the AON is an oligoribonucleotide.

8. The AON according to any one of claims 1 to 7, wherein the AON comprises at least one non-natural linkage modification.

9. The AON according to claim 8, wherein the non-natural linkage modification is a phosphorothioate linkage.

10. The AON according to any one of claims 1 to 9, comprising at least one nucleotide that is mono-, or disubstituted at the 2′, 3′ and/or 5′ position of the sugar moiety.

11. The AON according to claim 10, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; O-alkyl, S-alkyl, or N-alkyl; O-alkenyl, S-alkenyl, or N-alkenyl; O-alkynyl, S-alkynyl or N-alkynyl; O-allyl, S-allyl, or N-allyl; O-alkyl-O-alkyl; -methoxy; -aminopropoxy; -methoxyethoxy; -dimethylaminooxyethoxy; and -dimethylaminoethoxyethoxy.

12. The AON according to claim 11, wherein
(a) all sugar moieties are modified with a 2′-O-methyl (2′-OMe) substitution, or wherein all sugar moieties are modified with a 2′-methoxyethoxy (2′-MOE) substitution;
(b) the 5′- and 3′-terminal nucleotides are LNA-modified, and all other sugar moieties are modified with a 2′-OMe substitution or a 2′-MOE substitution; or
(c) the two 5′- and two 3′-terminal nucleotides are LNA-modified, and all other sugar moieties are modified with a 2′-OMe substitution or a 2′-MOE substitution.

13. A viral vector comprising a nucleotide sequence encoding an AON according to any one of claims 1 to 6.

14. A pharmaceutical composition comprising an AON according to any one of claims 1 to 12, or a viral vector according to claim 13, and further comprising one or more of a carrier, excipient, stabilizer, transfection agent, diluent, gelling agent or buffer.

15. An AON according to any one of claims 1 to 12 for use in the treatment of a human subject suffering from dystrophic epidermolysis bullosa (DEB).

16. The AON for use according to claim 15, wherein the treatment comprises topical administration of the AON.

17. The AON for use according to claim 16, wherein the AON is in a carbomer hydrogel formulation.

18. Use of an AON according to any one of claims 1 to 12 or a viral vector according to claim 13 in the manufacture of a medicament for the treatment, prevention, amelioration or delay of DEB in a human subject.

19. A method of preventing or reducing exon 105 inclusion into a human COL7A1 mRNA when the mRNA is produced by splicing from a pre-mRNA in a human cell, the method comprising providing the cell in an in vitro, in vivo or ex vivo setting, and administering to the cell an AON according to any one of claims 1 to 12, a viral vector according to claim 13, or a pharmaceutical composition according to claim 14.

20. A method for the treatment of DEB in a human subject, comprising the step of administering to the subject an AON according to any one of claims 1 to 12, a viral vector according to claim 13, or a pharmaceutical composition according to claim 14.

21. The AON for use of any one of claims 15 to 17, the use of claim 18, or the method of claim 20, wherein the DEB is caused by a mutation in exon 105 of a COL7A1 gene in the human subject.

22. The AON for use of any one of claims 15 to 17, the use of claim 18, or the method of claim 20, wherein the DEB is caused by a c.7864delC mutation in exon 105 of a COL7A1 gene in the human subject.
